(19) United States
(12) Patent Application Publication
(10) Pub. No.: US 2025/0186325 A1
(43) Pub. Date: Jun. 12, 2025

(54) COMPOSITIONS OF ASCORBIC ACID SALTS

(71) Applicant: SHISEIDO COMPANY, LTD., Tokyo (JP)

(72) Inventors: Binhua YANG, East Windsor, NJ (US); Vincent CHO, East Windsor, NJ (US)

(21) Appl. No.: 18/535,483

(22) Filed: Dec. 11, 2023

(51) Int. Cl. A61K 8/55; A61K 8/06; A61K 8/23; A61K 8/34; A61K 8/44; A61K 8/60; A61K 8/81; A61K 8/85; A61Q 19/00

(52) U.S. Cl. CPC A61K 8/55; A61K 8/062; A61K 8/23; A61K 8/34; A61K 8/44; A61K 8/602; A61K 8/8152; A61K 8/85; A61Q 19/00; A61K 2800/10; A61K 2800/51; A61K 2800/522; A61K 2800/524

(57) ABSTRACT

The present application relates to emulsions of ascorbic acid salts. In some embodiments, a composition includes water and oil. The composition includes two or more ascorbic acid derivatives in a total amount of about 9 wt % to about 15 wt %. The ascorbic acid derivatives are selected from the group consisting of sodium ascorbyl phosphate (SAP), magnesium ascorbyl phosphate (MAP), and ascorbyl glucoside (AA2G). The composition includes an emulsifier and a functional polymer.

### FIELD

The present disclosure is directed to compositions of ascorbic acid salts.

### BACKGROUND

Skin is the first line of defense against environmental insults that would otherwise damage sensitive underlying tissue and organs. For example, skin maintains a relatively water-impermeable barrier between an organism and its environment to prevent dehydration. Additionally, skin plays a key role in a person's physical appearance. Generally, most people desire to have young, healthy looking skin. As a result, treating the signs of aging in skin has become a booming business. Treatments range from cosmetic creams and moisturizers to various forms of cosmetic surgery.

As an example, due to its high solubility in aqueous media, advantageous physiological activities, and antioxidant capabilities, L-ascorbic acid has been used for various purposes, including cosmetics. L-ascorbic acid, however, is unstable in aqueous media because of its reducibility and is susceptible to undergo oxidative degradation, losing its physiological activity.

In particular, L-Ascorbic acid (or vitamin C), is an α-ketolactone. The number 2 and 3 carbons are double-bonded and contain an acid-ionizable hydrogen in water (pK=4.2). Ascorbic acid is also a moderately strong reductant. These properties, which lead to instability in the ascorbic acid structure, have been burdensome to pharmacologists when attempting to formulate active ascorbic acid compositions. Thus, at higher pH's, the ascorbic acid increasingly becomes the notoriously unstable ascorbate anion. This instability may be due to several causes not restricted to:
a) Stereochemical strain due to polar repulsive forces. Thus, when the 2-hydroxy group is the one to ionize, it places two negative charges (the other being on the carboxyl oxygen) in close proximity which favors ring disruption.
b) Oxidative degradation due to the ascorbate anion's propensity to act as a reductant. The one-electron oxidation product (dehydroascorbate free radical) tends to disproportionate, forming another ascorbate molecule and the two-electron oxidation product, dehydroascorbate, which is extremely unstable in aqueous solutions and breaks down to ultimately form species such as L-threonic acid and oxalic acid. Transition metal ions can catalyze these reactions.
c) Degradation due to bulk water attack. Thus at lower ascorbate concentrations or ionic strength, water itself can react with and degrade the ascorbate molecule.

Accordingly, water-based cosmetic compositions including L-ascorbic acid as a component are typically not stable. Eliminating water by using waxes, oils, or silicone materials from the composition cures this problem. However, the solubility of L-ascorbic acid in non-aqueous media is poor and use of waxes, oils, or silicone materials can form a waxy or oily residue on the skin that does not evaporate.

Other attempts to provide stable compositions of ascorbic acid have included addition of a urea agent, which can provide unpleasant odor to a composition in addition to skin irritation when in high concentrations. In addition, the ascorbic acid is in particle form, which can promote localized hot spots of the L-ascorbic acid on the skin, which promotes skin irritation.

As an alternative to L-ascorbic acid, a number of L-ascorbic acid derivatives have been tested to allow use of compounds having physiological activity but without instability in aqueous media. However, high (efficacious) concentration of such L-ascorbic acid derivatives promotes crystallization of the derivatives from the aqueous media, reducing the efficacy of the derivatives and, like the L-ascorbic acid itself, promoting localized hot spots of the L-ascorbic acid derivatives on the skin, which promotes skin irritation.

In addition, U.S. Pat. No. 9,949,917 and U.S. Publication No. 2005/0220726 disclose cosmetic compositions containing sucrose fatty acid esters, such as sucrose laurate, sucrose dilaurate and sucrose trilaurate for use as skin lightening agents. However, when skin care compositions are provided in the form of an emulsion, these sucrose fatty acid esters may destabilize the emulsion due to their emulsifying properties due to the hydrophilic and lipophilic moieties in these compounds. Emulsion instability may manifest in a variety of ways (e.g., flocculation, creaming, sedimentation, or coalescence), all of which are generally undesirable in a skin care composition.

Therefore, there is a need for ascorbic acid-based emulsions that are storage stable for extended periods of time with reduced or eliminated degradation and/or crystallization of the ascorbic acid-based compounds while maintaining or improving the efficacy of the ascorbic acid-based compounds.

References for citing in an Information Disclosure Statement (37 C.F.R. 1.97(h)). U.S. Pat. Nos. 6,124,348; 8,765,416; 11,654,100; 11,337,899; 8,053,469; 5,140,043; 5,308,621; U.S. 2023/0338244; 2021/0077365; 2022/0047471; 2022/0183936; 2021/0228467; EP 4008406.

### SUMMARY

The present application relates to emulsions of ascorbic acid salts.

In some embodiments, a composition includes water and oil. The composition includes two or more ascorbic acid derivatives in a total amount of about 9 wt % to about 15 wt %. The ascorbic acid derivatives are selected from the group consisting of sodium ascorbyl phosphate (SAP), magnesium ascorbyl phosphate (MAP), and ascorbyl glucoside (AA2G). The composition includes an emulsifier and a functional polymer.

### BRIEF DESCRIPTION OF THE FIGURES

The patent or application file contains at least one drawing executed in color. Copies of this patent or patent application publication with color drawing will be provided by the Office upon request and payment of the necessary fee.

So that the manner in which the above recited features of the present disclosure can be understood in detail, a more particular description of the disclosure, briefly summarized above, may be had by reference to aspects, some of which are illustrated in the appended drawings. It is to be noted, however, that the appended drawings illustrate only typical aspects of this present disclosure and are therefore not to be considered limiting of its scope, for the present disclosure may admit to other equally effective aspects.

FIG. 1 is a graph illustrating thickening efficiency of compositions, according to some embodiments.

FIG. 2 depicts photos illustrating color and appearance changes of compositions at 0° C. or 25° C. over time, according to some embodiments.

FIG. 3 depicts photos illustrating color and appearance changes of compositions at 37° C. or 40° C. over time, according to some embodiments.

FIG. 4 is a graph illustrating composition viscosity increase with increased pH value, according to some embodiments.

FIG. 5 depicts photos illustrating color and appearance changes of compositions at 0° C. and 25° C. over time, according to some embodiments.

FIG. 6 depicts photos illustrating color and appearance changes of compositions at 37° C. and 40° C. over time, according to some embodiments.

FIG. 7 is a graph illustrating viscosity increase with concentration increase of Aristoflex Silk, according to some embodiments.

FIG. 8 depicts photos illustrating color and appearance changes of compositions at 0° C. and 25° C. over time, according to some embodiments.

FIG. 9 depicts photos illustrating color and appearance changes of compositions at 37° C. and 40° C. over time, according to some embodiments.

FIG. 10 is a graph illustrating viscosity versus NET-WRS concentration, according to some embodiments.

FIG. 11 is a graph illustrating pH versus viscosity of compositions, according to some embodiments.

FIG. 12 depicts photos illustrating color and appearance changes after two weeks at varying temperatures, according to some embodiments.

FIG. 13 depicts photos illustrating color and appearance changes of compositions after four (4) weeks at varying temperatures, according to some embodiments.

To facilitate understanding, identical reference numerals have been used, where possible, to designate identical elements that are common to the figures. The figures are not drawn to scale and may be simplified for clarity. It is contemplated that elements and features of one aspect may be beneficially incorporated in other aspects without further recitation.

### DETAILED DESCRIPTION

The present disclosure is directed to compositions of ascorbic acid derivatives. Compositions of the present disclosure can be oil-in-water emulsions including two or more ascorbic acid derivatives, an emulsifier, and a functional polymer. Compositions of the present disclosure can be emulsions that are storage stable for extended periods of time with reduced or eliminated degradation and/or crystallization of the ascorbic acid-based compounds while maintaining or improving the efficacy of the ascorbic acid-based compounds.

It has been discovered that the combination of a hydrophilic emulsifier (such as a copolymer of the ester of methacrylic acid and Beheneth-25) and a salt-tolerant functional polymer (such as a taurate polymer) can provide compositions having efficacious amounts of ascorbic acid derivatives (e.g., 10 wt % or greater of ascorbic acid derivatives) without degradation of the ascorbic acid derivative(s) but with maintained stabilization of an oil-in-water emulsion (e.g., substantially no or no biphasic separation of the emulsion) and without crystallization (localized hot spots) of ascorbic acid derivative(s) during use.

For example, compositions of the present disclosure can provide efficacious use of oil-in-water emulsions without skin irritation. In addition, use of a functional polymer that is salt-tolerant (e.g., of iron) reduces or prevents degradation (depolymerization) of the functional polymer during use which promotes the stabilization of the oil-in-water emulsion.

In addition, compositions of the present disclosure may be substantially free of or entirely free of silicone, polyethylene glycol, alcohol, fragrance, or combinations thereof. Thus, when applied to the skin, component(s) of compositions of the present disclosure can evaporate from the skin without leaving a waxy or oil residue left behind.

### Compositions

Compositions of the present disclosure can include two or more ascorbic acid derivatives (e.g., L-ascorbic acid derivatives), one or more emulsifiers, and one or more functional polymers. Compositions can be formed using any suitable method such as by mixing the two or more ascorbic acid derivatives, the one or more emulsifiers, and the one or more functional polymers with water and/or oil and combining all components together. For example, ascorbyl glucoside can be mixed with buffer solution (e.g., citric acid and sodium citrate) followed by introducing the buffer solution having ascorbyl glucoside to the remainder components of the emulsion.

The compositions of the present disclosure can be structured or formulated into a variety of different forms. Non-limiting examples include emulsions (e.g., oil-in-water) in the forms of creams, lotions, serums, gels, and masks. Variations and other structures will be apparent to the skilled artisan and are appropriate for use in the present disclosure. Lotions and creams are generally structured as oil-in-water emulsions. As used herein, an “oil-in-water emulsion” includes an oil as a dispersed phase that is distributed into water as a continuous phase. The dispersed phase is intruded in the continuous phase. Dispersed versus continuous phases can be controlled by which component (oil or water) is the major component (greater wt %) and which component is the minor component (lesser wt %). For oil-in-water emulsions, oil is the minor component and water is the major component. Toners, serums, and cleansers can be aqueous solutions, gels, or oil-in-water emulsions. One difference between toners, serums, and cleansers when compared with lotions and creams is that the former tend to be less viscous than the latter.

In some embodiments, a composition can be an oil-in-water emulsion.

In some embodiments, the composition includes a total amount of sodium ascorbyl phosphate (SAP), magnesium ascorbyl phosphate (MAP), ascorbyl glucoside (AA2G), or combinations thereof of about 9 wt % to about 15 wt %, such as about 10 wt % to about 15 wt %, such as about 10.5 wt % to about 15 wt %, such as about 11 wt % to about 14 wt %, such as about 11 wt % to about 13 wt %, alternatively about 10 wt % to about 11 wt %, alternatively about 11 wt % to about 12 wt %, alternatively about 12 wt % to about 13 wt %, alternatively about 13 wt % to about 14 wt %, alternatively about 14 wt % to about 15 wt %, based on the weight of the composition.

In some embodiments, the composition includes an ascorbyl glucoside (AA2G) in an amount of about 0.05 wt % to about 3 wt %, such as about 1 wt % to about 3 wt %, such as about 2 wt % to about 3 wt %, alternatively about 0.1 wt % to about 1 wt %, such as about 0.1 wt % to about 0.5 wt %, alternatively about 0.5 wt % to about 0.9 wt %, such as about 0.1 wt % to about 0.2 wt %, alternatively about 0.2 wt % to about 0.3 wt %, alternatively about 0.3 wt % to about 0.4 wt %, alternatively about 0.4 wt % to about 0.5 wt %, alternatively about 0.5 wt % to about 0.6 wt %, alternatively about 0.7 wt % to about 0.8 wt %, alternatively about 0.8 wt % to about 0.9 wt %, alternatively about 0.9 wt % to about 1 wt %, based on the weight of the composition.

In some embodiments, the composition includes a magnesium ascorbyl phosphate (MAP) in an amount of about 0.05 wt % to about 3.5 wt %, such as about 0.1 wt % to about 1 wt %, such as about 1 wt % to about 2 wt %, alternatively about 2 wt % to about 3 wt %, such as about 0.1 wt % to about 0.5 wt %, alternatively about 0.5 wt % to about 1 wt %, alternatively about 1 wt % to about 1.5 wt %, alternatively about 1.5 wt % to about 2 wt %, alternatively about 2 wt % to about 2.5 wt %, alternatively about 2.5 wt % to about 3 wt %, alternatively about 3 wt % to about 3.5 wt %, based on the weight of the composition. It has been discovered that an amount of MAP above about 3 wt % or above about 3.5 wt % can promote pH imbalance.

In some embodiments, the composition includes a sodium ascorbyl phosphate (SAP) in an amount of about 0.05 wt % to about 14 wt %, such as about 0.1 wt % to about 10 wt %, such as about 5 wt % to about 9.5 wt %, alternatively about 5 wt % to about 6 wt %, such as about 6 wt % to about 7 wt %, alternatively about 7 wt % to about 8 wt %, alternatively about 8 wt % to about 9 wt %, alternatively about 9 wt % to about 10 wt %, alternatively about 10 wt % to about 11 wt %, alternatively about 11 wt % to about 12 wt %, alternatively about 12 wt % to about 13 wt %, based on the weight of the composition.

In some embodiments, the composition includes a functional polymer in an amount of about 0.05 wt % to about 3.5 wt %, such as about 0.1 wt % to about 3.5 wt %, such as about 1 wt % to about 3.5 wt %, such as about 1 wt % to about 2 wt %, such as about 1.25 wt % to about 1.75 wt %, alternatively about 1 wt % to about 1.5 wt %, alternatively about 1.25 wt % to about 1.5 wt %, alternatively about 1.5 wt % to about 1.75 wt %, alternatively about 1.75 wt % to about 2 wt %, based on the weight of the composition.

In some embodiments, the composition includes an emulsifier in an amount of about 1 wt % to about 7 wt %, such as about 2 wt % to about 6 wt %, such as about 2.5 wt % to about 5 wt %, alternatively about 2 wt % to about 3 wt %, alternatively about 3 wt % to about 4 wt %, alternatively about 4 wt % to about 5 wt %, based on the weight of the composition.

In some embodiments, the composition includes an additional ascorbic acid derivative (in addition to SAP, AA2G, and MAP) in an amount of about 0.05 wt % to about 2 wt %, such as about 0.1 wt % to about 1.5 wt %, such as about 0.1 wt % to about 1 wt %, alternatively about 0.1 wt % to about 0.3 wt %, alternatively about 0.3 wt % to about 0.5 wt %, alternatively about 0.5 wt % to about 0.7 wt %, alternatively about 0.7 wt % to about 0.9 wt %, alternatively about 0.9 wt % to about 1.1 wt %, alternatively about 1.1 wt % to about 1.3 wt %, alternatively about 1.3 wt % to about 1.5 wt %, alternatively about 1.5 wt % to about 2 wt %, based on the weight of the composition.

Compositions of the present disclosure can include the components of the composition (e.g., AA2G, functional polymer, emulsifier, etc.) and/or reaction product(s) of two or more of the components of the composition. Claims directed to compositions of the present disclosure embrace such reaction product(s), if any such reaction product(s) might be present.

Components of compositions of the present disclosure (e.g., AA2G, functional polymer, emulsifier, etc.) can be purchased from commercial source(s) or obtained synthetically via any suitable process known in the art.

Compositions of the present disclosure can have a pH of about 5.5 to about 7.5, such as about 5.5 to about 5.8, alternatively about 6.5 to about 7.5. For example, in some embodiments, a composition has two or more ascorbic acid derivatives (such as SAP and AA2G) and has a pH of about 6.5 to about 7.5. In some embodiments, a composition has three ascorbic acid derivatives (such as SAP, MAP, and AA2G) and has a pH of about 5.5 to about 5.8. It has been discovered that a pH of about 5.5 to about 5.8 can reduce or eliminate MAP crystallization.

Compositions of the present disclosure can be substantially free (e.g., about 1 wt % or less) of or entirely free of silicone, polyethylene glycol, alcohol (ethanol), fragrance, or combinations thereof. In some embodiments, a composition can have about 1 wt % or less, such as about 0 wt %, or about 0.1 wt % to about 0.3 wt % of silicone, polyethylene glycol, alcohol, fragrance, or combinations thereof.

Compositions of the present disclosure can be oil-in-water emulsions. For example, compositions can include water (as a water phase) in an amount of about 50 wt % to about 99 wt % water, such as about 75 wt % to about 95 wt %, such as about 75 wt % to about 85 wt %, alternatively about 85 wt % to about 95 wt %, such as about 85 wt % to about 90 wt %, based on the total weight of the composition. Compositions can include oil (as an oil phase) in an amount of about 1 wt % to about 25 wt %, such as about 5 wt % to about 25 wt %, such as about 5 wt % to about 15 wt %, such as about 7 wt % to about 13 wt %, such as about 9 wt % to about 11 wt %, such as about 10.5 wt %, alternatively about 15 wt % to about 25 wt %, such as about 15 wt % to about 20 wt %, alternatively about 20 wt % to about 25 wt %.

In some embodiments, an oil is selected from isodecyl neopentanoate, neopentyl glycol diheptanoate, coco-caprylate/caprate, squalene, tocopheryl acetate, and combinations thereof. For example, a composition has about 2 wt % to about 4 wt % isodecyl neopentanoate, about 1 wt % to about 3 wt % neopentyl glycol diheptanoate, about 0.01 wt % to about 1.1 wt % coco-caprylate/caprate, about 2 wt % to about 4 wt % squalene, about 0.3 wt % to about 1 wt % tocopheryl acetate, or combinations thereof.

In some embodiments, an oil is selected from squalene, ethylhexyl olivate, and combinations thereof. For example, a composition has about 5 wt % to about 15 wt % of squalene+ethylhexyl olivate, such as about 7 wt % to about 13 wt %, such as about 9 wt % to about 11 wt %.

In some embodiments, an oil can include a hydrocarbon, an ester oil, a vegetable fat/oil, a higher alcohol, and a higher fatty acid.

Examples of the hydrocarbon include liquid paraffin, paraffin, squalane, and squalene.

Examples of the ester oil include isopropyl myristate, cetyl octanoate, octyldodecyl myristate, isopropyl palmitate, butyl stearate, cetyl lactate, myristyl lactate, isocetyl stearate, isocetyl isostearate, triisostearate, cetyl 2-ethylhexanoate, pentaerythritol tetra2-ethylhexanoate, glycerin tri2-ethylhexanoate, 2-ethylhexyl palmitate, and oleyl oleate.

Examples of the vegetable oil/fat include but are not limited to avocado oil, camellia oil, macadamia nut oil, corn oil, olive oil, rapeseed oil, sesame oil, castor oil, peanut oil, almond oil, soybean oil, tea seed oil, jojoba oil, and germ oil.

Examples of higher alcohol include oleyl alcohol, stearyl alcohol, isostearyl alcohol, behenyl alcohol, octyldodecanol, decyltetradecanol, jojoba alcohol, cetyl alcohol, and myristyl alcohol, and examples of the higher fatty acid include oleic acid, isostearic acid, linolic acid, linoleic acid, eicosapentaenoic acid, palmitic acid, stearic acid, and behenic acid.

### Ascorbic Acid Derivatives

Ascorbic acid derivatives of the present disclosure can include sodium ascorbyl phosphate (SAP), magnesium ascorbyl phosphate (MAP), ascorbyl glucoside (AA2G), or combinations thereof.

In some embodiments, a composition includes an effective amount of an ascorbyl compound (e.g., sodium ascorbyl phosphate, magnesium ascorbyl phosphate, or ascorbyl glucoside) to inhibit melanin production in melanocytes, to reduce tyrosinase activity in skin, to reduce oxidative damage in skin caused by free radicals, to increase collagen-1 expression in skin which can help with reducing the appearance of wrinkles and fine lines and reduce the appearance of sagging or non-elastic skin, to increase lysyl oxidase expression in skin, which can help with reducing the appearance of wrinkles and fine lines and reduce the appearance of sagging or non-elastic skin by increasing cross-linking of collagens and elastin, and/or reduce expression of TNF-α and lipoxygenase in skin, which can help reduce skin inflammation (e.g., reddened or erythemic skin, sensitive skin, etc.). In some embodiments, the composition includes an effective amount of sodium ascorbyl phosphate (SAP), magnesium ascorbyl phosphate (MAP), ascorbyl glucoside (AA2G), or combinations thereof to decrease tyrosinase activity in the skin, decrease melanin production in the skin, reduce oxidation of the skin, increase collagen-1 expression in the skin, increase lysyl oxidase in the skin, and/or reduce TNF-α and lipoxygenase activity in the skin.

Two or more different types of ascorbic acid derivatives can be utilized in compositions of the present disclosure. One is sodium ascorbyl phosphate, which has the following chemical structure:

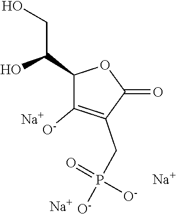 [SMILES: O=C1O[C@H]([C@@H](O)CO)C([O-])=C1CP(=O)([O-])[O-] ; [Na+] ; [Na+] ; [Na+]]

Sodium ascorbyl phosphate is commercially available from a variety of sources. A non-limiting example of a commercial supplier of sodium ascorbyl phosphate is DSM (Switzerland) STAY-C® 50 or SODIUM ASCORBYL PHOSPHATE supplied by BASF

Alternatively, or additionally, magnesium ascorbyl phosphate can be utilized, which has the following chemical structure:

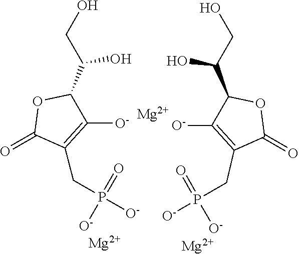 [SMILES: O=C1O[C@H]([C@@H](O)CO)C([O-])=C1CP(=O)([O-])[O-] ; [Mg+2] ; O=C1O[C@H]([C@@H](O)CO)C([O-])=C1CP(=O)([O-])[O-] ; [Mg+2] ; [Mg+2]]

Magnesium ascorbyl phosphate is commercially available from a variety of sources. A non-limiting example of a commercial supplier of magnesium ascorbyl phosphate is RONACARE® MAP available from EMD Performance Materials of Philadelphia, Pennsylvania.

Alternatively, or additionally, ascorbyl glucoside can be utilized. The ascorbyl glucoside can be 1-ascorbic acid 2-glucoside, which can have the following structure:

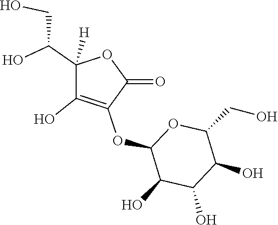 [SMILES: O=C1O[C@H]([C@H](O)CO)C(O)=C1O[C@H]1O[C@H](CO)[C@@H](O)[C@H](O)[C@H]1O]

For example, the ascorbyl glucoside can be purchased from Hayashibara Company (Japan) under the trade name AA2G™. Sodium ascorbyl phosphate has the ability to reduce melanin production in melanocytes and reduce tyrosinase activity, which can be beneficial to treating melanogenesis or hyperpigmentation in skin. Sodium ascorbyl phosphate has anti-oxidative properties and can act as an antioxidant to free radicals or reactive oxygen species. Sodium ascorbyl phosphate has the ability to increase collagen-1 expression, which can be beneficial in reducing the appearance of fine lines or wrinkles or reducing the appearance of sagging or non-elastic skin. Also, sodium ascorbyl phosphate has the ability to increase lysyl oxidase in skin, which can be beneficial in reducing the appearance of fine lines or wrinkles or reducing the appearance of sagging or non-elastic skin by increasing cross-linking of elastins and collagens, thereby creating a more structurally sound matrix of supportive proteins in the skin. Sodium ascorbyl phosphate has the ability to reduce both TNF-alpha and lipoxygenase activity in skin, which can be beneficial in treating inflamed skin.

In some embodiments, the additional ascorbic acid derivative (in addition to MAP, SAP, and AA2G) is selected from ascorbic acid polypeptide, ascorbyl dipalmitate, ascorbyl methylsilanol pectinate, ascorbyl palmitate, ascorbyl stearate, disodium ascorbyl sulfate, esters of ascorbic acid, methylsilanol ascorbate, potassium ascorbyl tocopheryl phosphate, and combinations thereof.

### Emulsifiers

Compositions of the present disclosure include one or more emulsifiers. Emulsifiers can reduce the interfacial tension between phases and improve the composition and stability of an emulsion.

In some embodiments, an emulsifier is a copolymer of acrylate and/or methacrylate monomeric units. For example, the copolymer can be a beheneth-25 methacrylate copolymer. Beheneth-25 is a polyethylene glycol ether. The copolymer can be the copolymer of an ester of methacrylic acid and Beheneth-25.

In some embodiments, an emulsifier includes a copolymer, a stearate, a glycol, a polypropylene glycol copolymer, a phenoxyalcohol, or combinations thereof.

In some embodiments, an emulsifier includes a copolymer, a tetraisostearate, dipropylene glycol, sorbitan sesquiisostearate, a polypropylene glycol copolymer (such as PPG-8-ceteth-20), a phenoxyethanol, or combinations thereof.

In some embodiments, Net-WRS is included in a composition of the present disclosure. Net-WRS is manufactured by Barnet Products Corp. of Englewood Cliffs. N.J. The Net-WRS is a mixture of Sorbeth-30 Tetraisostearate (PEG-30 Sorbitol Tetraisostearate), sorbitan sesquiisostearate, PPG-8 Deceth-20 (a polyoxypropylene, polyoxyethylene ether of cetyl alcohol), acrylates/beheneth-25 methacrylate copolymer, dipropylene glycol and water. This composition for cosmetic use is a thickening polymeric gel with lipophilic and hydrophilic portions that can form a matrix. Sorbeth-30 is a polyethylene glycol ether of sorbitol with an average of 30 moles of ethylene oxide.

Compositions of the present disclosure can include one or more additional emulsifiers. Additional emulsifiers can include nonionic emulsifiers (see, for example, U.S. Pat. Nos. 5,011,681; 4,421,769; and 3,755,560). Examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, polyethylene glycol 20 sorbitan monolaurate (polysorbate 20), polyethylene, steareth-2, steareth-20, steareth-21, ceteareth-20, cetearyl glucoside, cetearyl alcohol, ceteth-10, polysorbate 80, cetyl phosphate, potassium cetyl phosphate, polysorbate 60, glyceryl stearate, or combinations thereof.

In some embodiments, an additional emulsifier is one or more medium to long chain fatty-acid derivative emulsifier (e.g., about 12-20 carbon chain, alternatively 16-20 carbon chain). In some examples, the emulsifier may include a non-ionic, medium to long chain fatty-acid derivative emulsifier, such as steareth-2 steareth-21, glycereth-25 pyrrolidonecarboxylic acid isostearate, and combinations of these. “Steric acid-derived emulsifier” refers to an emulsifier in which at least one of the lipophilic domains of the surfactant is comprised of a saturated 18-carbon chain (similar to stearic acid). These emulsifiers typically contain stearate, steareth, or isostearate in their chemical names and are often derived from stearic acid combined with other chemical moieties. Particularly suitable emulsifiers include stearic-acid derived emulsifiers with a hydrophilic-lipophilic balance (HLB) of about 10 to about 12. An additional emulsifier may be present in the composition at 0.05 wt % to 5 wt % (e.g., 0.1 wt % to 4 wt %, 0.5 wt % to 3 wt % or even 1 wt % to 2 wt %), based on the total weight of the composition. In some instances, it may be desirable to exclude certain polyether modified silicone emulsifiers, such a PEG-11 methyl ether dimethicone, PEG-12 dimethicone, PEG/PPG 19/19 dimethicone, or other PEGylated dimethicones, which may destabilize an oil-in-water emulsion.

### Functional Polymers

Functional polymers of the present disclosure can be any suitable salt-tolerant rheology modifying polymer.

In some embodiments, a functional polymer is a hydrophobically modified aqueous rheology modifier (e.g., thickening agent) to provide the composition with suitable rheological, stability, and skin feel properties. Some non-limiting examples of rheology modifier that may be suitable for use herein may include crosslinked polyacrylate polymers, such as sodium polyacryloyldimethyl taurate such as ARISTOFLEX SILK sold by Clariant.

In some embodiments, a functional polymer is comprising at least 5 mol-% of units (a) according to Formula (1):

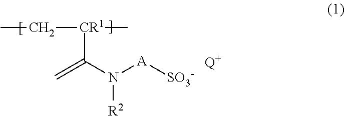

wherein:
R¹ and R² are independently selected from H, methyl, or ethyl; A is a linear or branched C₁-C₁₂-alkylene group; and Q+ is a cosmetically acceptable cation. The polymer of Formula (1) may be synthesized according to any suitable polymerization method known in the art or, in some embodiments, can be obtained commercially under the tradename ARISTOFLEX® from CLARIANT®, such as ARISTOFLEX® SILK (INCI name SODIUM POLYACRYLOYLDIMETHYL TAURATE).

The polymer may be crosslinked or non-crosslinked. In at least one embodiment, the polymer is a crosslinked or non-crosslinked homopolymer.

In at least one embodiment, the polymer has a weight average molecular weight of at least 700 g/mol, such as about 700 g/mol to about 10 million g/mol.

In at least one embodiment, the polymer comprises or consists of:
(a) from 5 to 99.99 mol-% repeating units (a) according to Formula (1);
(b) from 0.01 to 10 mol-% crosslinking or branching units (b), wherein the crosslinking or branching units (b) result from the incorporation of a monomer comprising at least two olefinically unsaturated double bonds;
(c) optionally from 0 to 89 mol-% of neutral structural units (c);
(d) optionally from 0 to 20 mol-% of anionic structural units (d), wherein the anionic structural units (d) result from the incorporation of a monomer comprising at least one carboxylate anion.

### Repeating Units (a)

In at least one embodiment, the polymer comprises at least 9 mol-% of repeating units (a) according to Formula (1). In at least one embodiment, the polymer comprises at least 9.49 mol-% of repeating units (a) according to Formula (1). In at least one embodiment, the polymer comprises from 5 to 100 mol-%, or from 90 to 99.9 mol-%, or from 95 to 99.5 mol-%, or from 27.5 to 97.4 mol-%, or from 40 to 99 mol-%, or from 55 to 98 mol-%, or from 9 to 99.9 mol-%, or from 9.49 to 99.8 mol-%, or from 9.49 to 98 mol-%, or from 10 to 99.7 mol-%, or from 20 to 99.5 mol-%, or from 30 to 99 mol-%, or from 40 to 98 mol-%, or from 40 to 95 mol-%, or from 50 to 90 mol-%, or from 60 to 80 mol-%, or from 45 to 75 mol-%, of repeating units (a) according to Formula (1). In at least one embodiment, the polymer consists of repeating units (a) according to Formula (1).

Optionally, repeating units (a) according to Formula (1) may partly or completely comprise a bio-based carbon content. In at least one embodiment, at least 10 wt %, such as at least 20 wt % of the repeating units (a) according to Formula (1) comprise from 28 wt % to 100 wt % bio-based carbon content, relative to the total mass of carbon in the repeating unit (a) according to Formula (1), measured according to standard ASTM D6866-12, Method B.

In at least one embodiment, at least 30 wt %, or at least 40 wt %, or at least 50 wt %, or at least 60 wt %, or at least 70 wt %, or at least 80 wt %, or at least 90 wt %, or at least 95 wt %, or at least 98 wt %, or at least 99 wt % of the repeating units (a) according to Formula (1) comprise from 28 wt % to 100 wt % bio-based carbon content, relative to the total mass of carbon in the repeating unit (a) according to Formula (1), measured according to standard ASTM D6866-12, Method B. In at least one embodiment, the repeating unit (a) according to Formula (1) comprises at least 35 wt %, such as at least 40 wt %, such as at least 54 wt %, such as at least 57 wt %, such as about 100 wt % bio-based carbon content, relative to the total mass of carbon in the repeating unit (a) according to Formula (1), measured according to standard ASTM D6866-12, Method B.

Q⁺ of Formula (1) may be any cosmetically acceptable cation. In at least one embodiment, Q⁺ is H⁺, NH₄⁺, an organic ammonium ion [NHR⁵R⁶R⁷]⁺ wherein R⁵, R⁶, and R⁷ independently of one another may be hydrogen, a linear or branched alkyl group having 1 to 22 carbon atoms, a linear or branched, singularly or multiply unsaturated alkenyl group having 2 to 22 carbon atoms, a C₆-C₂₂ alkylamidopropyl group, a linear mono-hydroxyalkyl group having 2 to 12 carbon atoms or a linear or branched dihydroxyalkyl group having 3 to 12 carbon atoms, and where at least one of the radicals R⁵, R⁶, and R⁷ is not hydrogen, or Q⁺ is Li⁺, Na⁺, K⁺, ½ Ca⁺⁺, ½ Mg⁺⁺, ½ Zn⁺⁺, ⅓ Al⁺⁺⁺, or combinations thereof.

In at least one embodiment, residue A in Formula (1) is selected from the group consisting of methylene, ethylene, n-propylene, —CH(CH₃)—CH₂—, —C(CH₃)₂—CH₂—, —C(CH₃)₂—(CH₂)ₙ—, and —CHCH₃—(CH₂)ₙ—, wherein n is 1, 2, 3 or an integer between 4 and 12.

In at least one embodiment, the polymer comprises repeating units (a) according to Formula (1) wherein R¹ and R² are independently selected from H, methyl or ethyl; A is a linear or branched C₁-C₁₂-alkylene group; and Q⁺ is H⁺, NH₄⁺, Li⁺, Na⁺, K⁺, ½ Ca⁺⁺, ½ Mg⁺⁺, ½ Zn⁺⁺, ⅓ Al⁺⁺⁺, or combinations thereof, such as wherein Q⁺ is Na⁺ or NH₄⁺. NH₄⁺ may be preferred because it is more soluble in the favored solvent used in the polymer synthesis. Na⁺ may be preferred because of reduced likelihood of unpreferred gases being produced during synthesis and also due to economic advantages.

In at least one embodiment, Q⁺ is NH₄⁺. In at least one embodiment, Q⁺ is Na⁺. In at least one embodiment, Q⁺ is selected from the group monoalkylammonium, dialkylammonium, trialkylammonium and/or tetraalkylammonium salts, in which the alkyl substituents of the amines may independently of one another be (C₁ to C₂₂)-alkyl radicals or (C₂ to C₁₀)-hydroxyalkyl radicals.

In at least one embodiment, the polymer comprises at least one repeating unit (a) according to Formula (1), for example one repeating unit (a) according to Formula (1). In at least one embodiment, the polymer comprises two or more different repeating units (a) according to Formula (1), for example repeating units according to Formula (1) having different Q⁺ counterions.

In at least one embodiment, the repeating units (a) according to Formula (1) have a degree of neutralization of between 0 mol % and 100 mol %. In at least one embodiment, the repeating units (a) according to Formula (1) have a degree of neutralization of from 50 to 100 mol %, such as from 80 to 100 mol %, such as from 90 to 100 mol %, such as from 95 to 100 mol %. In some embodiments, a degree of neutralization of more than 80 mol %, such as more than 90 mol-%, such as more than 95 mol %.

In at least embodiment, the repeating units (a) according to Formula (1) result from the incorporation of a monomer selected from the group consisting of acryloyldimethyltaurates, acryloyl-1,1-dimethyl-2-methyltaurates (ACDMT), acryloyltaurates, acryloyl-N-methyltaurates, and combinations thereof. In some embodiments, the repeating units (a) according to Formula (1) result from the incorporation of acryloyldimethyltaurate.

For example, the repeating units according to Formula (1) are formed by polymerization of a compound according to Formula (3):

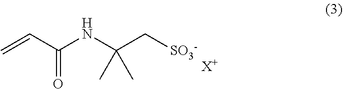

wherein X is a proton.

The polymer may be crosslinked or non-crosslinked. The polymer may be branched or unbranched. In at least one embodiment, the polymer is crosslinked and/or branched. In at least one embodiment, the polymer comprises, in addition to repeating units (a) according to Formula (1), crosslinking or branching units (b), wherein the crosslinking or branching units (b) result from the incorporation of a monomer comprising at least two olefinically unsaturated double bonds. In at least one embodiment, the polymer comprises from 0.01 to 10 mol %, or from 0.01 to 5 mol %, or from 0.01 to 4 mol %, or from 0.01 to 3 mol %, of crosslinking or branching units (b), wherein the crosslinking or branching units (b) result from the incorporation of a monomer comprising at least two olefinically unsaturated double bonds.

In at least one embodiment, the polymer comprises (or consists of):
(a) from 90 to 99.9 mol %, such as from 95 to 99.5 mol % of repeating units (a) according to Formula (1); and
(b) from 0.01 to 10 mol %, such as from 0.01 to 5 mol % of crosslinking or branching units (b).

In at least one embodiment, the polymer comprises from 96 to 99.7 mol %, such as from 97 to 99.5 mol % units (a) and from 0.3 to 4 mol %, such as from 0.5 to 3 mol % units (b). In at least one embodiment, the polymer comprises units (a) and (b), such that the sum thereof is at least 99 mol %, by total weight of the polymer.

In at least one embodiment, the polymer comprises or consists of repeating units (a) resulting from the incorporation of a monomer selected from the group consisting of acryloyldimethyltaurates, acryloyl-1,1-dimethyl-2-methyltaurates (ACDMT), acryloyltaurates, acryloyl-N-methyltaurates, and combinations thereof, in particular wherein the polymer comprises or consists of repeating units (a) resulting from the incorporation of acryloyldimethyltaurate.

### Crosslinking or Branching Units (b)

In at least one embodiment, the polymer comprises crosslinking or branching units (b), wherein the crosslinking or branching units result from the incorporation of a monomer comprising at least two olefinically unsaturated double bonds. In at least one embodiment, the polymer comprises from 0.01 to 5 mol %, such as from 0.01 to 4 mol %, such as from 0.01 to 3 mol %, even such as from 0.01 to 2 mol % of crosslinking or branching units.

In at least one embodiment, the crosslinking or branching units comprise at least one oxygen, nitrogen, sulfur, or phosphorus atom. In at least one embodiment, the crosslinking or branching units result from monomers having a molecular weight of less than 500 g/mol. In at least one embodiment, the units (b) result from bifunctional or trifunctional crosslinking agents.

In at least one embodiment, the polymer comprises two or more different crosslinking or branching units.

In at least one embodiment, the crosslinking or branching units result from the incorporation (into the backbone structure of the polymer) of a monomer according to Formula (2):

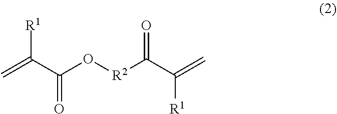

R¹ is selected from H, methyl or ethyl;
R² is a linear or branched alkyl group having 1 to 6 carbon atoms, or is a linear or branched, mono- or polyunsaturated alkylene group having 2 to 6 carbon atoms, or a group —(CH₂—CH₂—O)ₙ—; and n is an integer from 1 to 100.

Monomers according to Formula (2) have the advantage that polymers based thereon can be more brush-like. However, brush-like polymers show different properties, versus linear ones. For example, depending on the different comonomer units the solubility can be in- or decreased.

In at least one embodiment, the crosslinking or branching units are according to Formula (4):

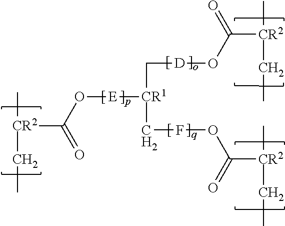

wherein:
R¹ is selected from H, methyl or ethyl;
R² is a linear or branched alkyl group having 1 to 6 carbon atoms, hydrogen, or a linear or branched, mono- or polyunsaturated alkylene group having 2 to 6 carbon atoms;
D, E, and F are independently methyleneoxy (—CH₂O—), ethyleneoxy (—CH₂—CH₂—O—), propyleneoxy (—CH(CH₃)—CH₂—O—), a linear or branched alkylene group having 1 to 6 carbon atoms, a linear or branched, singularly or multiply unsaturated alkenylene group having 2 to 6 carbon atoms, a linear mono-hydroxyalkylene group having 2 to 6 carbon atoms or a linear or branched dihydroxyalkylene group having 3 to 6 carbon atoms; and
o, p, and q each independently is an integer from 1 to 50.

Crosslinking or branching units according to Formula (4) have the advantage that polymers comprising those can be highly branched.

In at least one embodiment, the crosslinking or branching units result from the incorporation of a monomer selected from the group consisting of methylenebisacrylamide; methylenebismethacrylamide; esters of unsaturated monocarboxylic and polycarboxylic acids with polyols, such as di-acrylates and tri-acrylates and -methacrylates (e.g. glycerol propoxylate triacrylate [GPTA]), such as butanediol and ethylene glycol diacrylate and polyethylene glycol diacrylate and -methacrylate, trimethylolpropane triacrylate (TMPTA) and trimethylolpropane trimethacrylate (TMPTMA); allyl compounds, such as allyl (meth)acrylate, triallyl cyanurate, diallyl maleate, polyallyl esters, tetraallyloxyethane, triallylamine, tetraallylethylenediamine; allyl esters of phosphoric acid; and/or vinylphosphonic acid derivatives. The choice of crosslinking or branching units is important in view of the flexibility of the crosslinks between the main chains of the polymer, which affects the final performance of the polymer.

In at least one embodiment, the crosslinking or branching units result from the incorporation of a crosslinker selected from trimethylolpropane triacrylate (TMPTA) and/or glycerol propoxylate triacrylate (GPTA). In some embodiments, crosslinkers are glycerol propoxylate triacrylate (GPTA), trimethylolpropane triacrylate (TMPTA), pentaerythritol diacrylate mono stearate (PEAS), hexanediol diacrylate (HDDA), polyethylene glycol diacrylate (PEG-DA) or hexanediol dimethacrylate (HDDMA).

In at least one embodiment, the crosslinking or branching units (b) result from the incorporation of a crosslinker selected from trimethylolpropane triacrylate (TMPTA) and/or glycerol propoxylate triacrylate (GPTA).

In at least embodiment, the crosslinking or branching units (b) comprise at least 35 wt %, such as at least 40 wt %, such as at least 54 wt %, such as at least 57 wt %, such as about 100 wt % bio-based carbon content, relative to the total mass of carbon in the crosslinking or branching units (b), measured according to standard ASTM D6866-12, Method B.

### Neutral Structural Units (c)

In at least embodiment, the polymer comprises at least one neutral structural unit (c). In at least one embodiment, the polymer comprises one or more neutral structural units (c). In at least one embodiment, the polymer comprises from 0.01 to 88.52 mol %, or from 0.05 to 72.4 mol %, or from 0.99 to 59.99 mol %, or from 1.99 to 44.99 mol %, of neutral structural units.

In at least one embodiment, the polymer comprises at least one neutral structural unit (c) resulting from the incorporation of a monomer selected from the group consisting of acrylic or methacrylic acid amides, polyglycol acrylic or methacrylic acid esters, polyglycol acrylic or methacrylic acid amides, ethoxylated fatty alcohol acrylates, ethoxylated fatty alcohol methacrylates, propoxylated fatty alcohol acrylates, and open-chain or cyclic N-vinylamides or N-methylvinyl amides.

In at least one embodiment, the polymer comprises at least one neutral structural unit (c) resulting from the incorporation of a monomer selected from the group consisting of open-chain N-vinyl amides, such as N-vinylformamide, N-vinylmethylformamide, N-vinylmethylacetamide or N-vinylacetamide; cyclic N-vinyl amides (N-vinyl lactams) with a ring size of 3 to 9, such as N-vinylpyrrolidone (NVP) or N-vinylcaprolactam; amides of acrylic and methacrylic acid; alkoxylated acrylamides and alkoxylated methacrylamides.

In at least one embodiment, the polymer comprises at least one neutral structural unit (c) resulting from the incorporation of a monomer selected from the group consisting of N-vinylformamide, N-vinylacetamide, N-methyl-N-vinylformamide, N-methyl-N-vinylacetamide, N-vinyl-2-pyrrolidone, N-vinylcaprolactam, vinylacetate, N,N-dimethylacrylamide, N-isopropylacrylamide, acrylamide, methylacrylate, behenylpolyethoxy-(25)-methacrylate, laurylpolyethoxy-(7)-methacrylate, cetylpolyethoxy-(10)-methacrylate, stearylpolyethoxy-(8)-methacrylate, methoxypolyethoxy-(12)-methacrylate, and combinations thereof.

In at least one embodiment, the polymer comprises at least one neutral structural unit (c) resulting from the incorporation of a monomer selected from the group consisting of hydroxyethyl methacrylate, hydroxymethyl methacrylamide, hydroxyethyl methacrylamide, hydroxypropyl methacrylamide, N,N-dimethylaminomethyl methacrylate and N,N-diethylaminomethyl methacrylate.

In at least one embodiment, the polymer comprises at least one neutral structural unit (c) resulting from the incorporation of a monomer selected from the group consisting of methylvinylether, ethylvinylether, methylallylether, ethylmethallylether, methylmethallylether, ethylallylether, tert-butylacrylamide, N,N-diethylacrylamide, N,N-dimethylacrylamide, N,N-dimethylmethacrylamide, N,N-dipropylacrylamide, N-isopropylacrylamide, N-propylacrylamide, acrylamide, methacrylamide, methylacrylate, methylmethacrylate, tert-butylacrylate, tert-butylmethacrylate, n-butylacrylate, n-butylmethacrylate, laurylacrylate, laurylmethacrylate, behenylacrylate, behenylmethacrylate, cetylacrylate, cetylmethacrylate, stearylacrylate, stearylmethacrylate, tridecylacrylate, and tridecylmethacrylate.

In at least one embodiment, the polymer comprises at least one neutral structural unit (c) resulting from the incorporation of a monomer selected from the group consisting of polyethoxy-(5)-methacrylate, polyethoxy-(5)-acrylate, polyethoxy-(10)-methacrylate, polyethoxy-(10)-acrylate, behenylpolyethoxy-(7)-methacrylate, behenylpolyethoxy-(7)-acrylate, behenylpolyethoxy-(8)-methacrylate, behenylpolyethoxy-(8)-acrylate, behenylpolyethoxy-(12)-methacrylate, behenylpolyethoxy-(12)-acrylate, behenylpolyethoxy-(16)-methacrylate, behenylpolyethoxy-(16)-acrylate, behenylpolyethoxy-(25)-methacrylate, behenylpolyethoxy-(25)-acrylate, laurylpolyethoxy-(7)-methacrylate, laurylpolyethoxy-(7)-acrylate, laurylpolyethoxy-(8)-methacrylate, laurylpolyethoxy-(8)-acrylate, laurylpolyethoxy-(12)-methacrylate, laurylpolyethoxy-(12)-acrylate, laurylpolyethoxy-(16)-methacrylate, laurylpolyethoxy-(16)-acrylate, laurylpolyethoxy-(22)-methacrylate, laurylpolyethoxy-(22)-acrylate, laurylpolyethoxy-(23)-methacrylate, laurylpolyethoxy-(23)-acrylate, cetylpolyethoxy-(2)-methacrylate, cetylpolyethoxy-(2)-acrylate, cetylpolyethoxy-(7)-methacrylate, cetylpolyethoxy-(7)-acrylate, cetylpolyethoxy-(10)-methacrylate, cetylpolyethoxy-(10)-acrylate, cetylpolyethoxy-(12)-methacrylate, cetylpolyethoxy-(12)-acrylate cetylpolyethoxy-(16)-methacrylate, cetylpolyethoxy-(16)-acrylate cetylpolyethoxy-(20)-methacrylate, cetylpolyethoxy-(20)-acrylate, cetylpolyethoxy-(25)-methacrylate, cetylpolyethoxy-(25)-acrylate, cetylpolyethoxy-(25)-methacrylate, cetylpolyethoxy-(25)-acrylate, stearylpolyethoxy-(7)-methacrylate, stearylpolyethoxy-(7)-acrylate, stearylpolyethoxy-(8)-methacrylate, stearylpolyethoxy-(8)-acrylate, stearylpolyethoxy-(12)-methacrylate, stearylpolyethoxy-(12)-acrylate, stearylpolyethoxy-(16)-methacrylate, stearylpolyethoxy-(16)-acrylate, stearylpolyethoxy-(22)-methacrylate, stearylpolyethoxy-(22)-acrylate, stearylpolyethoxy-(23)-methacrylate, stearylpolyethoxy-(23)-acrylate, stearylpolyethoxy-(25)-methacrylate, stearylpolyethoxy-(25)-acrylate, tridecylpolyethoxy-(7)-methacrylate, tridecylpolyethoxy-(7)-acrylate, tridecylpolythoxy-(10)-methacrylate, tridecylpolyethoxy-(10)-acrylate, tridecylpolyethoxy-(12)-methacrylate, tridecylpolyethoxy-(12)-acrylate, tridecylpolyethoxy-(16)-methacrylate, tridecylpolyethoxy-(16)-acrylate, tridecylpolyethoxy-(22)-methacrylate, tridecylpolyethoxy-(22)-acrylate, tridecylpolyethoxy-(23)-methacrylate, tridecylpolyethoxy-(23)-acrylate, tridecylpolyethoxy-(25)-methacrylate, tridecylpolyethoxy-(25)-acrylate, methoxypolyethoxy-(7)-methacrylate, methoxypolyethoxy-(7)-acrylate, methoxypolyethoxy-(12)-methacrylate, methoxypolyethoxy-(12)-acrylate, methoxypolyethoxy-(16)-methacrylate, methoxypolyethoxy-(16)-acrylate, methoxypolyethoxy-(25)-methacrylate, and methoxypolyethoxy-(25)-acrylate.

The neutral structural units (c) may, for example, also be 2-vinylpyridine, 4-vinylpyridine, glycidyl methacrylate, styrene, acetoxystyrene, acrylonitrile, vinyl chloride, vinylidene chloride, or tetrafluoroethylene.

In at least one embodiment, the neutral structural units (c) comprise at least 35 wt %, at least 40 wt %, such as at least 54 wt %, such as at least 57 wt %, such as about 100 wt % bio-based carbon content, relative to the total mass of carbon in the neutral structural units (c), measured according to standard ASTM D6866-12, Method B.

### Anionic Structural Units (d)

In at least one embodiment, the polymer comprises at least one anionic structural unit (d) which is different from repeating unit (a). In at least one embodiment, the polymer comprises one or more anionic structural units (d) which are different from repeating units (a). In at least one embodiment, the polymer further comprises one or more anionic structural units (d), wherein the anionic structural units (d) result from the incorporation of a monomer comprising at least one carboxylate anion. In at least one embodiment, the polymer comprises from 1.98 to 20 mol %, such as from 2.5 to 18 mol % of anionic structural units (d) which are different from repeating units (a).

In at least one embodiment, the anionic structural units (d) result from the incorporation of a monomer according to Formula (A):

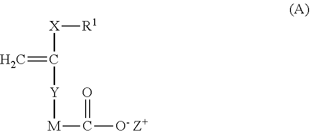

wherein:
R¹ and R³ are H, methyl or ethyl, or C(O)O⁻Z⁺;
X, Y are selected from a covalent bond, O, CH₂, C(O)O, OC(O), C(O)NR₃ or NR₃C(O);
M is selected from a covalent bond, —[C(O)O—CH₂—CH₂]ₙ—, a linear or branched alkylene group with 1 to 6 carbon atoms, a linear or branched, mono- or polyunsaturated alkenylene group with 2 to 6 carbon atoms, a linear mono-hydroxyalkylene group with 2 to 6 carbon atoms or a linear or branched dihydroxyalkylene group with 3 to 6 carbon atoms;
n is an integer from 1 to 5; and
Z⁺ is H⁺, NH₄⁺, an organic ammonium ion [HNR⁵R⁶R⁷]⁺;
wherein R⁵, R⁶ and R⁷ are independently hydrogen, a linear or branched alkyl group with 1 to 22 carbon atoms, a linear or branched, mono- or polyunsaturated alkenyl group with 2 to 22 carbon atoms, a C6 to C22 alkylamidopropyl group, a linear mono-hydroxyalkyl group with 2 to 10 carbon atoms or a linear or branched dihydroxyalkyl group with 3 to 10 carbon atoms, and wherein at least one of R⁵, R⁶ and R⁷ is not hydrogen, or Z⁺ is Li⁺, Na⁺, K⁺, ½ Ca⁺⁺, ½ Mg⁺⁺, ½ Zn⁺⁺, ⅓ Al⁺⁺⁺, or combinations thereof. In at least one embodiment, Z⁺ is H⁺, NH₄⁺, Li⁺, Na⁺, K⁺, ½ Ca⁺⁺, ½ Mg⁺⁺, ½ Zn⁺⁺, or ⅓ Al⁺⁺⁺, such as H⁺, NH₄⁺, Li⁺, Na⁺ or K⁺.

In at least one embodiment, the anionic structural units (d) result from the incorporation of a monomer according to Formula (A) wherein X is a covalent bond or CH₂. In at least one embodiment, the anionic structural units (d) result from the incorporation of a monomer according to Formula (A) wherein Y is a covalent bond, CH₂, C(O)O, or C(O)NR³. In at least one embodiment, the anionic structural units (d) result from the incorporation of a monomer according to Formula (A) wherein M is a covalent bond, —[C(O)O—CH₂—CH₂]n-, or a linear or branched alkylene group with 1 to 6 carbon atoms. In at least one embodiment, the anionic structural units (d) result results from the incorporation of a monomer according to Formula (A) wherein R¹ is H, methyl or ethyl; X is a covalent bond or CH₂; Y is a covalent bond, CH₂, C(O)O, or C(O)NR³; R³ is H, methyl or ethyl; M is a covalent bond, —[C(O)O—CH₂—CH₂]n-, or a linear or branched alkylene group with 1 to 6 carbon atoms; Z⁺ is H⁺, NH₄⁺, Li⁺, Na⁺, K⁺, ½ Ca⁺⁺, ½ Mg⁺⁺, ½ Zn⁺⁺, or ⅓ Al⁺⁺⁺, or combinations thereof.

In at least one embodiment, the polymer comprises at least one anionic structural unit (d) resulting from the incorporation of a monomer selected from the group consisting of acrylic acid or acrylate, methacrylic acid or methacrylate, itaconic acid or itaconate, carboxyethylacrylic acid or carboxyethylacrylate, carboxyethylacrylic acid oligomers or carboxyethylacrylate oligomers, 2-propylacrylic acid or 2-propylacrylate, 2-ethylacrylic acid or 2-ethylacrylate, and their respective alkali or alkaline earth metal salts.

In at least one embodiment, the polymer comprises at least one anionic structural unit (d) resulting from the incorporation of a monomer selected from the group consisting of acrylic acid or acrylate, methacrylic acid or methacrylate, itaconic acid or itaconate, carboxyethylacrylic acid or carboxyethylacrylate, carboxyethylacrylic acid oligomers or carboxyethylacrylate oligomers, and their respective alkali or alkaline earth metal salts. These anionic structural units may be preferred because they can easily be synthesized from bio-based sources.

In at least one embodiment, the polymer comprises at least one anionic structural unit (d) resulting from the incorporation of a monomer selected from the group consisting of acrylic acid, ammonium acrylate, sodium acrylate, potassium acrylate, lithium acrylate, zinc acrylate, calcium acrylate, magnesium acrylate, zirconium acrylate, methacrylic acid, ammonium methacrylate, sodium methacrylate, potassium methacrylate, lithium methacrylate, calcium methacrylate, magnesium methacrylate, zirconium methacrylate, zinc methacrylate, 2-carboxyethylacrylate, ammonium 2-carboxyethylacrylate, sodium 2-carboxyethylacrylate, potassium 2-carboxyethylacrylate, lithium 2-carboxyethylacrylate, zinc 2-carboxyethylacrylate, calcium 2-carboxyethylacrylate, magnesium 2-carboxyethylacrylate, zirconium 2-carboxyethylacrylate, 2-carboxyethylacrylate-oligomers, ammonium 2-carboxyethylacrylate-oligomers, sodium 2-carboxyethylacrylate-oligomers, potassium 2-carboxyethylacrylate-oligomers, lithium 2 carboxyethylacrylate-oligomers, zinc 2-carboxyethylacrylate-oligomers, calcium 2-carboxyethylacrylate-oligomers, magnesium 2-carboxyethylacrylate-oligomers, zirconium 2-carboxyethylacrylate-oligomers, itaconic acid, sodium itaconate, potassium itaconate, lithium itaconate, calcium itaconate, magnesium itaconate, zirconium itaconate, and zinc itaconate.

The anionic structural units (d) may, for example, result from the incorporation of 2-ethylacrylic acid, ammonium 2-ethylacrylate, sodium 2-ethylacrylate, potassium 2-ethylacrylate, lithium 2-ethylacrylate, calcium 2-ethylacrylate, magnesium 2-ethylacrylate, zirconium 2-ethylacrylate, zinc 2-ethylacrylate, 2-propylacryl acid, ammonium 2-propylacrylate, sodium 2-propylacrylate, potassium 2-propylacrylate, lithium 2-propylacrylate, calcium 2-propylacrylate, magnesium 2-propylacrylate, magnesium 2-propylacrylate, zirconium 2-propylacrylate, or zinc 2-propylacrylate.

The anionic structural units (d) may, for example, result from the incorporation of maleic acid, fumaric acid, crotonic acid, or senecic acid. The anionic structural units (d) may, for example, also be mono[2-(methacryloyloxy)ethyl]succinate or acrylamido- or methacrylamidoglycolic acid.

In at least one embodiment, the anionic structural units (d) comprise at least 35 wt %, such as at least 40 wt %, such as at least 54 wt %, such as at least 57 wt %, such as about 100 wt % bio-based carbon content, relative to the total mass of carbon in the anionic structural units (d), measured according to standard ASTM D6866-12, Method B.

Optionally, the polymer comprises one or more further repeating units (e). For example, an optional further repeating unit (e) may result from the incorporation of styrenesulfonic acid.

In at least one embodiment, the polymer comprises (or consists of):
(a) from 40 to 99 mol % or from 40 to 98 mol %, such as from 55 to 98 mol % of repeating units according to Formula (1);
(b) from 0.01 to 5 mol %, such as from 0.01 to 3 mol % of crosslinking or branching units (b), wherein the crosslinking or branching units (b) result from the incorporation of a monomer comprising at least two olefinically unsaturated double bonds; and
(c) from 0.99 to 59.99 mol %, such as from 1.99 to 44.99 mol % of neutral structural units.

In at least one embodiment, the polymer comprises from 45 to 97 mol %, such as from 65 to 96 mol % units (a), from 0.25 to 4 mol %, such as from 0.3 to 3 mol % units (b), from 2 to 54 mol %, such as from 2.5 to 34.5 mol % units (c). In at least one embodiment, the polymer comprises units (a), (b) and (c) such that the sum thereof is at least 99 mol %, by total weight of the polymer.

In at least one embodiment, the polymer comprises from 70 to 98 mol %, such as from 73 to 96 mol % units (a), from 0.6 to 2.5 mol %, such as from 0.75 to 2 mol % units (b), from 1.4 to 54 mol %, such as from 2.5 to 34.5 mol % units (c). In at least one embodiment, the polymer comprises units (a), (b) and (c) such that the sum thereof is at least 99 mol %, by total weight of the polymer.

In at least one embodiment, the polymer further comprises one or more neutral structural units and one or more anionic structural units (d) which are different from repeating units (a), wherein the anionic structural units (d) result from the incorporation of a monomer comprising at least one carboxylate anion.

In at least one embodiment, the polymer comprises:
from 0.01 to 88.52 mol %, such as from 0.05 to 72.4 mol % of neutral structural units; and from 1.98 to 20 mol %, such as from 2.5 to 18 mol % of anionic structural units (d) different from repeating units (a).

In at least one embodiment, the polymer comprises (or consists of):
(a) from 9.49 to 98 mol %, such as from 27.5 to 97.4 mol % repeating units according to Formula (1);
(b) from 0.01 to 5 mol-%, such as from 0.01 to 4 mol % crosslinking or branching units (b), wherein the crosslinking or branching units (b) result from the incorporation of a monomer comprising at least two olefinically unsaturated double bonds;
(c) from 0.01 to 88.52 mol %, such as from 0.05 to 72.4 mol % of neutral structural units; and
(d) from 1.98 to 20 mol %, such as from 2.5 to 18 mol % of anionic structural units (d), wherein the anionic structural units (d) result from the incorporation of a monomer comprising at least one carboxylate anion.

In at least one embodiment, the polymer comprises from 37 to 96.4 mol %, such as from 43 to 95.3 mol % units (a), from 0.1 to 3 mol %, such as from 0.2 to 2 mol % units (b), from 0.1 to 59.3 mol %, such as from 0.5 to 52.8 mol % units (c), and from 3.5 to 16 mol %, such as from 4 to 14 mol % units (d). In at least one embodiment, the polymer comprises units (a), (b), (c) and (d) such that the sum thereof is at least 99 mol %, by total weight of the polymer.

In at least one embodiment, the polymer comprises from 70 to 94.5 mol %, units (a), from 0.35 to 1.5 mol %, units (b), from 0.65 to 25.65 mol % units (c), and from 4.5 to 12 mol % units (d). In at least one embodiment, the polymer comprises units (a), (b), (c) and (d) such that the sum thereof is at least 99 mol %, by total weight of the polymer.

In at least one embodiment, the polymer is non-crosslinked. For example, the polymer may optionally consist of repeating units (A) of Formula (1).

### Composition Properties

### Storage Stability

Compositions of the present disclosure are capable of maintaining an emulsion when stored at room temperature for 12 months or longer. Such emulsion stability can be determined by visually observing whether or not a biphasic composition forms at 12 months or longer.

In addition, compositions of the present disclosure are capable of maintaining at least 90% of the starting content of ascorbic acid derivatives when the composition is stored at room temperature for 12 months or longer. Such storage stability is very advantageous for high concentration ascorbic acid derivative compositions of the present disclosure.

The amount of ascorbic acid derivative content in a composition can be determined using a wide range of techniques including, but not limited to: titrimetric, spectrophotometric, electrochemical, fluorimetric, enzymatic and chromatographic. Methods for determining ascorbic acid derivative content in a topical composition can be complicated/confounded by the presence of excipients or other antioxidant agents (e.g., agents for stabilizing Vitamin C), as well as degradation products. Of the above-listed methods, high performance liquid chromatography may be preferred. See, A M Maia et al., “Validation of HPLC stability-indicating method for Vitamin C in semisolid pharmaceutical/cosmetic preparations . . . ” Talanta Vol. 71, pp. 639-643 (2007).

In some embodiments, the storage stable compositions of this disclosure demonstrates less than 10 mol % degradation of the ascorbic acid derivatives after storage for 6 weeks or longer (e.g., 8 weeks or longer, 10 weeks or longer, 12 weeks or longer, 18 weeks or longer, 24 weeks or longer, or even longer) at 40° C.±2° C. in a sealed container, such as less than 9 mol %, less than 8 mol %, less than 7 mol %, less than 6 mol %, less than 5 mol %, less than 4 mol %, less than 3 mol %, less than 2 mol % degradation of the ascorbic acid derivatives initially present in the composition prior to storage.

In some embodiments, the storage stable compositions of this disclosure demonstrates less than 10 mol % degradation of the ascorbic acid derivatives after storage for 4 weeks or longer (e.g., 6 weeks or longer, 8 weeks or longer, 10 weeks or longer, 12 weeks or longer, 18 weeks or longer, 24 weeks or longer, or even longer) at 45° C.±2° C. in a sealed container, such as less than 9 mol %, less than 8 mol %, less than 7 mol %, less than 6 mol %, less than 5 mol %, less than 4 mol %, less than 3 mol %, less than 2 mol % degradation of the ascorbic acid initially present in the composition prior to storage.

In some embodiments, the storage stable compositions of this disclosure demonstrates less than 10 mol % degradation of the ascorbic acid derivatives after storage for 6 months or longer (e.g., 8 months or longer, 10 months or longer, 12 months or longer, 18 months or longer, or even longer) at 25° C.+2° C. in a sealed container or a multi-use container, such as less than 9 mol %, less than 8 mol %, less than 7 mol %, less than 6 mol %, less than 5 mol %, less than 4 mol %, less than 3 mol %, less than 2 mol % degradation of the ascorbic acid derivatives initially present in the composition prior to storage. In certain embodiments, the composition is stored in a sealed container. In certain embodiments, the composition is stored in a multi-use container.

In some embodiments, the storage stable compositions of this disclosure demonstrates less than 20 mol % degradation of the ascorbic acid after storage for 12 months or longer (e.g., 18 months or longer, 24 months or longer, or even longer) at 25° C.+2° C. in a sealed container or a multi-use container, such as less than 15 mol %, less than 12 mol %, less than 10 mol %, less than 8 mol %, less than 6 mol %, less than 6 mol %, less than 4 mol %, less than 3 mol %, less than 2 mol % degradation of the ascorbic acid derivatives initially present in the composition prior to storage. In certain embodiments, the composition is stored in a sealed container. In certain embodiments, the composition is stored in a multi-use container.

### Viscosity

In some embodiments, compositions of the present disclosure can have a viscosity of about 6,000 cPs or greater, such as about 6,300 cPs to about 12,000 cPs, such as about 6,500 cPs to about 7,500 cPs, such as about 6,500 cPs to about 7,500 cPs, such as about 7,500 cPs to about 8,500 cPs, such as about 8,500 cPs to about 9,500 cPs, such as about 9,500 cPs, such as about 9,500 cPs to about 10,500 cPs, such as about 10,500 cPs to 11,500 cps, alternatively from about 11,500 cPs to about 12,000 cPs.

### Additional Components

Compositions of the present disclosure may have one or more component(s) (also referred to herein as “ingredients”) in addition to the aforementioned components (ascorbic acid derivatives, functional polymer, and/or emulsifier). Additional component(s) may include cosmetic ingredients and/or pharmaceutically active ingredients. Some examples of these additional components are described in the following subsections.

### 1. Cosmetic Ingredients

The CTFA International Cosmetic Ingredient Dictionary and Handbook (2004 and 2008) describes a wide variety of non-limiting cosmetic ingredients that can be used in the context of the present disclosure. Examples of these ingredient classes include: fragrance agents (artificial and natural; e.g., gluconic acid, phenoxyethanol, and triethanolamine), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), flavoring agents/aroma agents (e.g., Stevia rebaudiana (sweetleaf) extract, and menthol), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as para-aminobenzoic acid (“PABA”) and corresponding PABA derivatives, titanium dioxide, zinc oxide, etc.), essential oils, vitamins (e.g., A, B, C, D, E, and K), trace metals (e.g., zinc, calcium and selenium), anti-irritants (e.g., steroids and non-steroidal anti-inflammatories), botanical extracts (e.g., Aloe vera, chamomile, cucumber extract, Ginkgo biloba, ginseng, and rosemary), anti-microbial agents, antioxidants (e.g., BHT and tocopherol), chelating agents (e.g., disodium EDTA, tetrasodium EDTA, tetrasodium glutamate diacetate), preservatives (e.g., potassium benzoate, sorbic acid, potassium sorbate, methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, methyl gluceth-20, saccharide isomerate, mannitol, glycerin, propanediol), exfoliants, waterproofing agents (e.g., magnesium/aluminum hydroxide stearate), skin conditioning agents (e.g., aloe extracts, allantoin, bisabolol, ceramides, hyaluronic acid, biosaccharide gum-1, ethylhexylglycerin, pentylene glycol, hydrogenated polydecene, octyldodecyl oleate, and dipotassium glycyrrhizate). Non-limiting examples of some of these ingredients are provided in the following subsections.

Compositions of the present disclosure can have a chelating agent in an amount of about 0.05 wt % to about 1 wt %, such as about 0.1 wt % to about 0.5 wt %, such as about 0.1 wt % to about 0.3 wt %.

Compositions of the present disclosure can have a humectant in an amount of about 1 wt % to about 15 wt %, such as about 2 wt % to about 4 wt %, alternatively about 9 wt % to about 11 wt %, alternatively about 12 wt % to about 14 wt %.

Compositions of the present disclosure can have a pH adjuster in an amount of about 0.5 wt % to about 2 wt %, such as about 0.5 wt % to about 1 wt %, alternatively about 1.5 wt % to about 2 wt %.

a. Moisturizing Agents

Non-limiting examples of moisturizing agents that can be used with the compositions of the present disclosure include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, polyglyceryl sorbitol, salts of pyrrolidone carboxylic acid, potassium PCA, propylene glycol, saccharide isomerate, sodium glucuronate, sodium PCA, sorbitol, sucrose, trehalose, urea, and xylitol.

Other examples include acetylated lanolin, acetylated lanolin alcohol, alanine, algae extract, Aloe barbadensis, Aloe barbadensis extract, Aloe barbadensis gel, Althea officinalis extract, apricot (Prunus armeniaca) kernel oil, arginine, arginine aspartate, Arnica montana extract, aspartic acid, avocado (Persea gratissima) oil, barrier sphingolipids, butyl alcohol, beeswax, behenyl alcohol, beta-sitosterol, birch (Betula alba) bark extract, borage (Borago officinalis) extract, butcherbroom (Ruscus aculeatus) extract, butylene glycol, Calendula officinalis extract, Calendula officinalis oil, candelilla (Euphorbia cerifera) wax, canola oil, caprylic/capric triglyceride, cardamom (Elettaria cardamomum) oil, carnauba (Copernicia cerifera) wax, carrot (Daucus carota sativa) oil, castor (Ricinus communis) oil, ceramides, ceresin, ceteareth-5, ceteareth-12, ceteareth-20, cetearyl octanoate, ceteth-20, ceteth-24, cetyl octanoate, cetyl palmitate, chamomile (Anthemis nobilis) oil, cholesterol, cholesterol esters, cholesteryl hydroxystearate, citric acid, clary (Salvia sclarea) oil, cocoa (Theobroma cacao) butter, coco-caprylate/caprate, coconut (Cocos nucifera) oil, collagen, collagen amino acids, corn (Zea mays) oil, fatty acids, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, Eucalyptus globulus oil, evening primrose (Oenothera biennis) oil, fatty acids, Geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (Vitis vinifera) seed oil, hazel (Corylus americana) nut oil, hazel (Corylus avellana) nut oil, hexylene glycol, hyaluronic acid, hybrid safflower (Carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (Jasminum officinale) oil, jojoba (Buxus chinensis) oil, kelp, kukui (Aleurites moluccana) nut oil, lactamide MEA, lanolin acetate, cetyl acetate, laneth-10 acetate, tocopheryl acetate, cellulose acetate propionate carboxylate, laneth-16, lanolin, lanolin acid, lanolin alcohol, lanolin oil, lanolin wax, lavender (Lavandula angustifolia) oil, lecithin, lemon (Citrus medica limonum) oil, linoleic acid, linolenic acid, Macadamia ternifolia nut oil, maltitol, matricaria (Chamomilla recutita) oil, methyl glucose sesquistearate, methylsilanol PCA, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (Olea europaea) oil, orange (Citrus aurantium dulcis) oil, palm (Elaeis guineensis) oil, palmitic acid, pantethine, panthenol, panthenyl ethyl ether, paraffin, PCA, peach (Prunus persica) kernel oil, peanut (Arachis hypogaea) oil, pentadecalactone, peppermint (Mentha piperita) oil, petrolatum, phospholipids, plankton extract, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinyl palmitate, rice (Oryza sativa) bran oil, RNA, rosemary (Rosmarinus officinalis) oil, rose oil, safflower (Carthamus tinctorius) oil, sage (Salvia officinalis) oil, sandalwood (Santalum album) oil, serine, serum protein, sesame (Sesamum indicum) oil, shea butter (Butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (Glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (Helianthus annuus) seed oil, sweet almond (Prunus amygdalus dulcis) oil, synthetic beeswax, tocopherol, tocopheryl linoleate, tribehenin, tridecyl neopentanoate, tridecyl stearate, triethanolamine, tristearin, urea, vegetable oil, water, waxes, wheat (Triticum vulgare) germ oil, and ylang (Cananga odorata) oil.

b. Antioxidants

Non-limiting examples of antioxidants that can be used with the compositions of the present disclosure include acetyl cysteine, ascorbic acid polypeptide, ascorbyl dipalmitate, ascorbyl methylsilanol pectinate, ascorbyl palmitate, ascorbyl stearate, BHA, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, phloretin, natural botanical antioxidants such as green tea or grape seed extracts, nordihydroguaiaretic acid, octyl gallate, phenylthioglycolic acid, potassium sulfite, propyl gallate, quinones, rosmarinic acid, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl linoleate, tocopheryl nicotinate, tocopheryl succinate, and tris(nonylphenyl)phosphite.

Compositions of the present disclosure can have an antioxidant in an amount of about 0.005 wt % to about 1 wt %, such as about 0.1 wt % to about 1 wt %, alternatively about 0.0075 wt % to about 0.05 wt %, such as about 0.0075 wt % to about 0.015 wt %.

c. Structuring Agents

In other non-limiting aspects, the compositions of the present disclosure can include a structuring agent. Structuring agent, in certain aspects, assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

d. Additional Emulsifiers

In certain aspects of the present disclosure, the compositions can include one or more additional emulsifiers (in addition to emulsifier(s) described above). Emulsifiers can reduce the interfacial tension between phases and improve the composition and stability of an emulsion. The emulsifiers can be nonionic, cationic, anionic, and zwitterionic emulsifiers (see, for example, U.S. Pat. Nos. 5,011,681; 4,421,769; and 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, DEA oleth-3 phosphate, polyethylene glycol 20 sorbitan monolaurate (polysorbate 20), polyethylene glycol 5 soya sterol, steareth-2, steareth-20, steareth-21, ceteareth-20, cetearyl glucoside, cetearyl alcohol, C12-13 pareth-3, PPG-2 methyl glucose ether distearate, PPG-5-ceteth-20, ceteth-10, polysorbate 80, cetyl phosphate, potassium cetyl phosphate, diethanolamine cetyl phosphate, polysorbate 60, glyceryl stearate, arachidyl alcohol, arachidyl glucoside, and mixtures thereof.

e. Silicone Containing Compounds

In non-limiting aspects, silicone containing compounds include any member of a family of polymeric products whose molecular backbone is made up of alternating silicon and oxygen atoms with side groups attached to the silicon atoms. By varying the Si—O chain lengths, side groups, and crosslinking, silicones can be synthesized into a wide variety of materials. They can vary in consistency from liquid to gel to solids.

The silicone containing compounds that can be used in the context of the present disclosure include those described in this specification or those known to a person of ordinary skill in the art. Non-limiting examples include silicone oils (e.g., volatile and non-volatile oils), gels, and solids. In certain aspects, the silicon containing compounds includes a silicone oils such as a polyorganosiloxane. Non-limiting examples of polyorganosiloxanes include dimethicone; polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, or eyes). A “volatile silicone oil” includes a silicone oil have a low heat of vaporization, i.e. normally less than about 50 cal per gram of silicone oil. Non-limiting examples of volatile silicone oils include: Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Mich. and dimethicone are described in the Third Edition of the CTFA Cosmetic Ingredient Dictionary (incorporated by reference) as and a mixture of fully methylated linear siloxane polymers end-blocked with trimethylsiloxy units, respectively. Other non-limiting volatile silicone oils that can be used in the context of the present disclosure include those available from General Electric Co., Silicone Products Div., Waterford, N.Y. and SWS Silicones Div. of Stauffer Chemical Co., Adrian, Mich.

f. Exfoliating Agent

Exfoliating agents include ingredients that remove dead skin cells on the skin's outer surface. These agents may act through mechanical, chemical, and/or other means. Non-limiting examples of mechanical exfoliating agents include abrasives such as pumice, silica, cloth, paper, shells, beads, solid crystals, solid polymers, etc. Non-limiting examples of chemical exfoliating agents include acids and enzyme exfoliants. Acids that can be used as exfoliating agents include, but are not limited to, glycolic acid, lactic acid, citric acid, alpha hydroxy acids, beta hydroxy acids, etc. Other exfoliating agents known to those of skill in the art are also contemplated as being useful within the context of the present disclosure.

g. Essential Oils

Essential oils include oils derived from herbs, flowers, trees, and other plants. Such oils are typically present as tiny droplets between the plant's cells, and can be extracted by several method known to those of skill in the art (e.g., steam distilled, effleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° C. to about 240° C. and densities ranging from about 0.759 to about 1.096.

Essential oils typically are named by the plant from which the oil is found. For example, rose oil or peppermint oil are derived from rose or peppermint plants, respectively. Non-limiting examples of essential oils that can be used in the context of the present disclosure include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang. Other essential oils known to those of skill in the art are also contemplated as being useful within the context of the present disclosure.

h. Additional Thickening Agents

Thickening agents (in addition to the functional polymer(s) described above), including thickeners or gelling agents, include substances which that can increase the viscosity of a composition. Thickeners include those that can increase the viscosity of a composition without substantially modifying the efficacy of the active ingredient within the composition. Thickeners can also increase the stability of the compositions of the present disclosure. In certain aspects of the present disclosure, thickeners include hydrogenated polyisobutene, trihydroxystearin, ammonium acryloyldimethyltaurate/VP copolymer, or a mixture of them.

Non-limiting examples of additional thickening agents that can be used in the context of the present disclosure include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see, for example, U.S. Pat. Nos. 5,087,445; 4,509,949; 2,798,053; and CTFA International Cosmetic Ingredient Dictionary, Fourth edition, 1991, pp. 12 and 80).

Non-limiting examples of cross-linked polyacrylate polymers include cationic and nonionic polymers. Non-limiting examples are described in U.S. Pat. Nos. 5,100,660; 4,849,484; 4,835,206; 4,628,078; and 4,599,379.

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (such as hydroxy ethylated or hydroxypropylated) to form a hydroxyalkylated cellulose which is then further modified with a C10-C30 straight chain or branched chain alkyl group through an ether linkage. Typically these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present disclosure can include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

Compositions of the present disclosure can have an additional thickening agent in an amount of about 0.05 wt % to about 1 wt %, such as about 0.1 wt % to about 0.5 wt %, such as about 0.2 wt % to about 0.4 wt %.

i. Preservatives

Non-limiting examples of preservatives that can be used in the context of the present disclosure include quaternary ammonium preservatives such as polyquaternium-1 and benzalkonium halides (e.g., benzalkonium chloride (“BAC”) and benzalkonium bromide), phenoxyethanol, benzyl alcohol, chlorobutanol, phenol, sorbic acid, potassium benzoate, potassium sorbate, thimerosal or combinations thereof.

Compositions of the present disclosure can have a preservative in an amount of about 0.05 wt % to about 2 wt %, such as about 0.5 wt % to about 1.5 wt %, such as about 0.8 wt % to about 1 wt %.

### Kits

Kits are also contemplated as being used in certain aspects of the present disclosure. For instance, compositions of the present disclosure can be included in a kit. A kit can include a container. Containers can include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of the composition. In other embodiments, the container can be squeezed (e.g., metal, laminate, or plastic tube) to dispense a desired amount of the composition. The containers can have pump or squeeze mechanisms. A kit can also include instructions for employing the kit components as well the use of any other compositions included in the container. Instructions can include an explanation of how to apply, use, and maintain the compositions.

### End Uses

In at least one embodiment, a composition of the present disclosure can be used in any suitable end use.

For example, the composition can be for use on skin.

In at least one embodiment, the composition is a skin moisturizing and/or nourishing composition.

In at least one embodiment, the composition is an emulsion such as an oil-in-water (o/w) emulsion, cream gel, or serum. In at least one embodiment, the composition is an oil-in-water emulsion.

### EXAMPLE EMBODIMENTS

The present disclosure provides, among others, the following embodiments, each of which can be considered as optionally including any alternate embodiments:

Clause 1. A composition comprising:
water;
an oil;
two or more ascorbic acid derivatives in a total amount of about 9 wt % to about 15 wt %, wherein the ascorbic acid derivatives are selected from the group consisting of sodium ascorbyl phosphate (SAP), magnesium ascorbyl phosphate (MAP), ascorbyl glucoside (AA2G); an emulsifier; and a functional polymer.

Clause 2. The composition of Clause 1, wherein the composition comprises the water in an amount of about 55 wt % to about 65 wt % and the oil in an amount of about 7 wt % to about 13 wt %.

Clause 3. The composition of Clauses 1 or 2, wherein the total amount of the two or more ascorbic acid derivatives is about 9 wt % to about 11 wt %.

Clause 4. The composition of any of Clauses 1 to 3, wherein the two or more ascorbic acid derivatives are SAP and AA2G, wherein SAP is present in an amount of about 8 wt % to about 10 wt % and AA2G is present in an amount of about 0.5 wt % to about 3 wt %.

Clause 5. The composition of any of Clauses 1 to 4, wherein the composition has a pH of about 6.5 to about 7.5.

Clause 6. The composition of any of Clauses 1 to 5, wherein the composition has about 2 wt % to about 4 wt % isodecyl neopentanoate, about 1 wt % to about 3 wt % neopentyl glycol diheptanoate, about 0.01 wt % to about 1.1 wt % coco-caprylate/caprate, about 2 wt % to about 4 wt % squalene, and about 0.3 wt % to about 1 wt % tocopheryl acetate.

Clause 7. The composition of any of Clauses 1 to 6, wherein the two or more ascorbic acid derivatives are SAP, AA2G, and MAP, wherein SAP is present in an amount of about 5 wt % to about 7 wt %, AA2G is present in an amount of about 0.5 wt % to about 3 wt %, and MAP is present in an amount of about 2 wt % to about 3.5 wt %.

Clause 8. The composition of any of Clauses 1 to 7, wherein the composition has a pH of about 5.5 to about 5.8.

Clause 9. The composition of any of Clauses 1 to 8, wherein the composition comprises about 7 wt % to about 13 wt % squalene+ethylhexyl olivate.

Clause 10. The composition of any of Clauses 1 to 19, wherein the functional polymer is present in an amount of about 1.25 wt % to about 1.75 wt %.

Clause 11. The composition of any of Clauses 1 to 10, wherein the emulsifier is present in an amount of about 2.5 wt % to about 5 wt %.

Clause 12. The composition of any of Clauses 1 to 11, wherein the composition has about 1 wt % or less of a combination of silicone, polyethylene glycol, and alcohol.

Clause 13. The composition of any of Clauses 1 to 12, wherein the emulsifier comprises a copolymer of (1) acrylate or methacrylate monomeric units and (2) a polyethylene glycol ether.

Clause 14. The composition of any of Clauses 1 to 13, wherein the copolymer is a copolymer of an ester of methacrylic acid and the polyethylene glycol ether.

Clause 15. The composition of any of Clauses 1 to 14, wherein the emulsifier includes the copolymer, a stearate, a glycol, a polypropylene glycol copolymer, and a phenoxyalcohol.

Clause 16. The composition of any of Clauses 1 to 15, wherein the emulsifier includes the copolymer, a tetraisostearate, dipropylene glycol, sorbitan sesquiisostearate, a polypropylene glycol copolymer, and a phenoxyethanol.

Clause 17. The composition of any of Clauses 1 to 16, wherein the polypropylene glycol copolymer is a polyethylene glycol ether of sorbitol with an average of about 30 moles of ethylene oxide.

Clause 18. The composition of any of Clauses 1 to 17, wherein the functional polymer comprises a polyacryloyldimethyl taurate.

Clause 19. The composition of any of Clauses 1 to 18, wherein the functional polymer comprises a sodium polyacryloyldimethyl taurate.

Clause 20. The composition of any of Clauses 1 to 19, wherein the composition further comprises:
a chelating agent in an amount of about 0.1 wt % to about 0.5 wt %;
a humectant in an amount of about 2 wt % to about 4 wt %, about 9 wt % to about 11 wt %, or about 12 wt % to about 14 wt %;
a pH adjuster in an amount of about 0.5 wt % to about 1 wt % or about 1.5 wt % to about 2 wt %;
an antioxidant in an amount of about 0.1 wt % to about 1 wt %; and
a preservative in an amount of about 0.5 wt % to about 1.5 wt %.

### EXAMPLES

### General Composition Formation

1. In the main container, mix water, humectants (such as Glycerin and Propanediol), preservative, EDTA, water thickener (such as siligel) and an oil in water emulsifier (NET-WRS) mix until all uniform.
2. In a side container, combine all the oil phase ingredients, mix until clear. Then add it to the water phase in the main container. Homogenize the batch until a uniform emulsion forms.
3. In another side container, make a buffer solution with Citric acid & Sodium Citrate and water. Then add AA2G in the buffer solution. Mix until uniform, add it to the main batch.
4. In another side container, premix SAP in water until all dissolve and add it to the main batch. Mix until uniform
5. Add polymer ARISTOFLEX® SILK to the main batch at the end, homogenize the batch until a uniform emulsion forms.

### Compositions Example 1

It is difficult to thicken the composition that contains 9% SAP and 1% AA2G. Three different polymeric thickeners were studied. They were used at same concentration 1.25% in the same composition. Each composition was prepared by mixing the ingredients at room temperature to form a homogenous o/w emulsion.

The stability of four 1000 ascorbic acid derivative compositions with different thickeners was measured and compared. The compositions are listed in Table 1.

TABLE 1
Composition of 10% Ascorbic Acid Derivative Compositions with Different Polymeric Thickeners

| Thickening Comparison of compositions | | | | | | |
|---|---|---|---|---|---|---|
| with different polymers | | | FL# 1 | FL# 2 | FL# 3 | FL# 4 |
| Function | Trade Name | US INCI | RM % | RM % | RM % | RM % |
| Solvent | DEIONIZED | WATER | 60.49 | 60.54 | 60.51 | 61.76 |
| | WATER | | | | | |
| Chelating | BIOPURE ™ | WATER, TETRASODIUM | 0.20 | 0.20 | 0.20 | 0.20 |
| | GLDA | GLUTAMATE DIACETATE, | | | | |
| | | SODIUM HYDROXIDE | | | | |
| Antioxidant | SODIUM | SODIUM METABISULFITE | 0.01 | 0.01 | 0.01 | 0.01 |
| | METABISULFITE | | | | | |
| Humectants | GLYCERINE | GLYCERIN | 3.00 | 3.00 | 3.00 | 3.00 |
| | 99.7%/MB | | | | | |
| | ZEMEA | PROPANEDIOL | 10.00 | 10.00 | 10.00 | 10.00 |
| Preservative | PHENOXETOL | PHENOXYETHANOL | 0.90 | 0.90 | 0.90 | 0.90 |
| | NF 272556 | | | | | |
| Thickener | SILIGEL ™ | XANTHAN GUM, | 0.30 | 0.30 | 0.30 | 0.30 |
| | | SCLEROTIUM | | | | |
| | | GUM, LECITHIN, | | | | |
| | | PULLULAN, SILICA | | | | |
| O/W | NET-WRS | SORBETH-30 | 2.50 | 2.50 | 2.50 | 2.50 |
| Emulsifier | | TETRAISOSTEARATE, | | | | |
| | | WATER, DIPROPYLENE | | | | |
| | | GLYCOL, SORBITAN | | | | |
| | | SESQUIISOSTEARATE, | | | | |
| | | ACRYLATES/BEHENETH-25 | | | | |
| | | METHACRYLATE COPOLYMER, | | | | |
| | | PPG-8-CETETH-20, | | | | |
| | | PHENOXYETHANOL | | | | |
| Oil | NEOLIGHT 100P | ISODECYL | 3.00 | 3.00 | 3.00 | 3.00 |
| | | NEOPENTANOATE | | | | |
| | LEXFEEL 7 | NEOPENTYL GLYCOL | 2.00 | 2.00 | 2.00 | 2.00 |
| | | DIHEPTANOATE | | | | |
| | VEGELIGHT | C9-12 ALKANE, COCO- | 2.00 | 2.00 | 2.00 | 2.00 |
| | C912-LC | CAPRYLATE/CAPRATE | | | | |
| | NEOSSANCE ™ | SQUALANE | 3.00 | 3.00 | 3.00 | 3.00 |
| | SQUALANE | | | | | |
| | VITAMIN E- | TOCOPHERYL ACETATE | 0.50 | 0.50 | 0.50 | 0.50 |
| | ACETATE CARE | | | | | |
| | 30499500 | | | | | |
| Buffer/pH | SODIUM | SODIUM | 0.080 | 0.080 | 0.080 | 0.080 |
| adjust | CITRATE | CITRATE | | | | |
| | CITRIC ACID | CITRIC ACID | 0.770 | 0.720 | 0.750 | 0.750 |
| | ANHYDROUS | | | | | |
| Ascorbic | Ascorbyl | Ascorbyl | 1.000 | 1.000 | 1.000 | 1.000 |
| acid | Glucoside | Glucoside | | | | |
| derivatives | Sodium Ascorbyl | Sodium Ascorbyl Phosphate | 9.000 | 9.000 | 9.000 | 9.000 |
| | Phosphate (salt) | | | | | |
| Polymer/ | Aristoflex Silk | Sodium Polyacryloyldimethyl | 1.250 | — | — | — |
| thickener | | Taurate | | | | |
| | Aristoflex AVC | Ammonium Acryloyldimethyl | — | 1.25 | — | — |
| | | Taurate/Vp Comolymer | | | | |
| | Sepinove EMT | HYDROXYETHYL | — | — | 1.25 | — |
| | | ACRYLATE/SODIUM | | | | |
| | | ACRYLOYLDIMETHYL | | | | |
| | | TAURATE COPOLYMER & | | | | |
| | | POLYSORBATE 60 & | | | | |
| | | SORBITAN ISOSTEARATE | | | | |

| | | | | |
|---|---|---|---|---|
| Total | 100.0000 | 100.0000 | 100.0000 | 100.0000 |

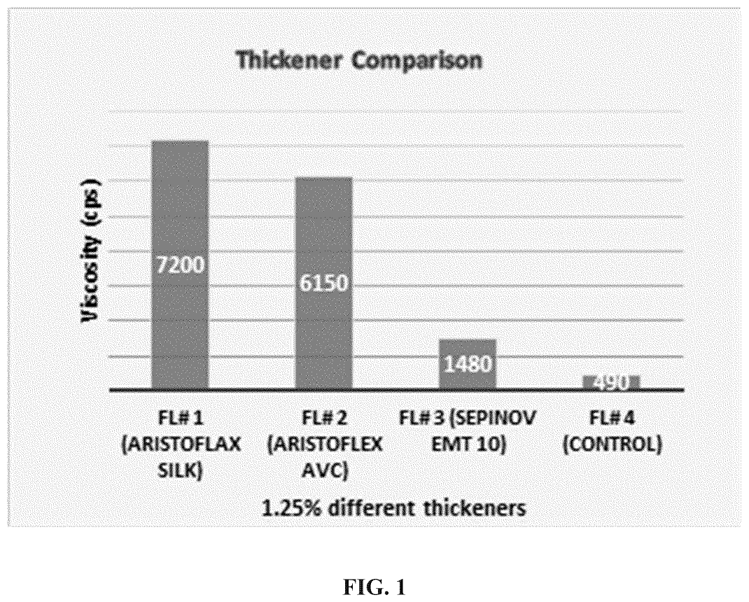

1.25% different polymeric thickener was used in each composition (FL #1, #2, #3), along with the control (FL #4 without any polymeric thickener). The pH of the compositions remained the same at 6.8. The viscosity and stability are shown in Table 2. The thickening efficiencies are compared in FIG. 1.

As can be seen, thickener selection has an important effect on the stability of the composition. Aristoflex Silk showed good thickening effect, as well as good stability at harsh conditions (3 cycles of Freeze/Thaw)

TABLE 2
Viscosity and Stability Comparison

| | | | Stability | Stability | Stability |
|---|---|---|---|---|---|
| | | Viscosity | @RT/ | @RT/ | @F/T |
| Formula# | Polymer | Cps | 1 week | 4 weeks | 3 cys |
| FL# 1 | Aristoflex Silk | 7200 | stable | stable | stable |
| FL# 2 | Aristoflex AVC | 6150 | stable | stable | unstable |
| FL# 3 | Sepinov EMT 10 | 1480 | unstable | unstable | unstable |
| FL# 4 | Control | 490 | unstable | unstable | unstable |

The compositions #1, 2, 3, and 4 were subjected to aging by placing them in an oven at 0° C., 25° C., 37° C. and 40° C. for four weeks. The degradation was evaluated in color change by visual inspection.

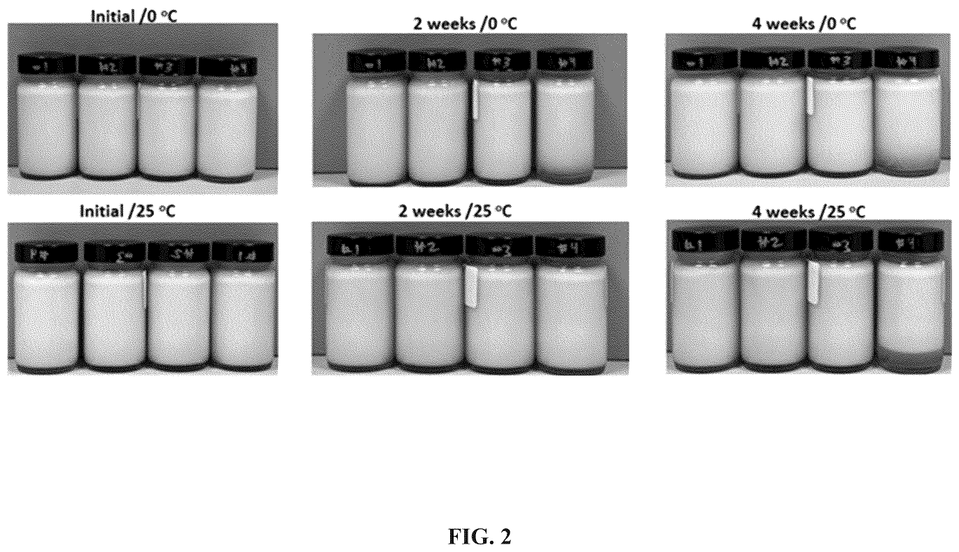

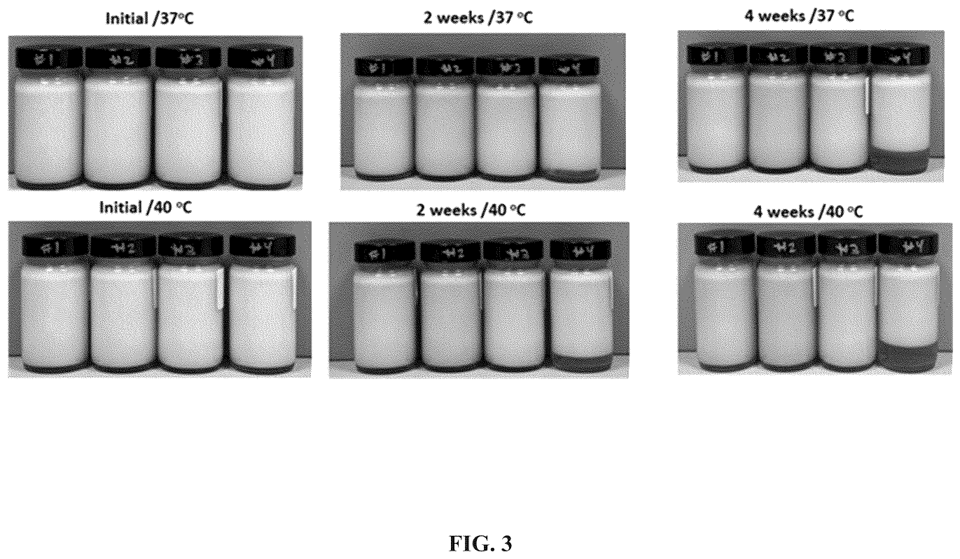

Pictures were taken at different stages, from initial, 2 weeks and 4 weeks. FIGS. 2 and 3 show the color and appearance changes in different temperature and over times. FIG. 2 is photos illustrating color and appearance changes of compositions at 0° C. or 25° C. over time. FIG. 3 is photos illustrating color and appearance changes of compositions at 37° C. or 40° C. over time. As shown in FIG. 2, minimal or no color change is observed with any of the compositions at 0° C. up to 4 weeks. However, composition #4 begins to phase separate at 2 weeks. At 4 weeks at 25° C., composition #4 has substantial phase separation. Very slight discoloration (from creamy white to brown/orange) was observed for compositions #1, #2, #3, and #4 beginning at 2 weeks at 25° C., and such discoloration became slightly more pronounced for each composition at 4 weeks at 25° C. As compared to discoloration at lower temperatures shown in FIG. 2, FIG. 3 shows discoloration for each composition that is more pronounced as temperature increased to 37° C. or 40° F. In addition, composition #4 became highly phase separated at 2 weeks and 4 weeks. Composition #1 had the least amount of discoloration (e.g., at 4 weeks at 40° C.) of any of compositions #1-#4, and composition #1 had no phase separation (e.g., at 4 weeks at 40° C.).

It is well known that vitamin C and ascorbic acid derivatives are not stable in high temperature. The color change to dark brown is an indication of the degradation of the vitamin C/ascorbic acid derivatives.

Aristoflex Silk was used in Formula #1 and showed the least color change even at 40° C. for 4 weeks.

### Compositions Example 2

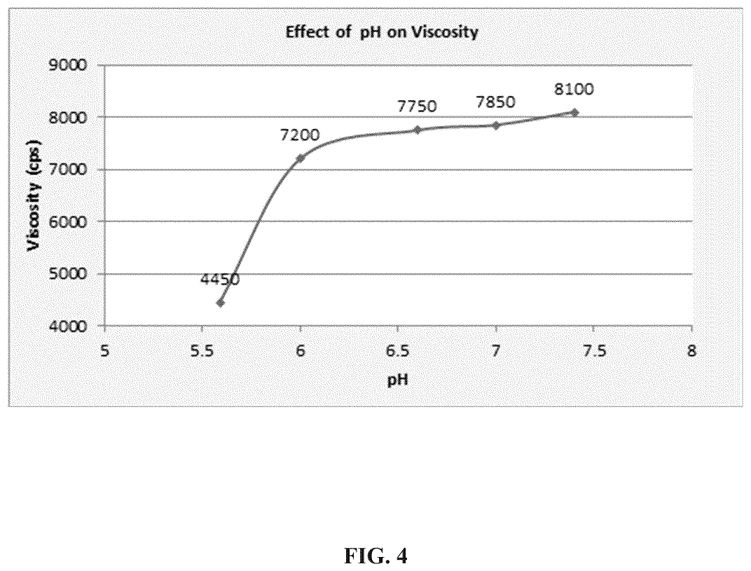

The pH value has significant impact on the ascorbic acid derivative stability of the compositions. Five compositions #5, 6, 7, 8, and 9 were prepared with same ingredients but varied pH values from 5.5 to 7.5, as adjusted by citric acid. The composition of the compositions are listed in Table 3. The viscosity and stability comparison showed in Table 4 and FIG. 4. FIG. 4 is a graph illustrating that composition viscosity increased with increased pH value. Desired viscosity was achieved when pH value>6. Table 4 shows that pH>6.5 results in a stable composition. Optimal pH range was between 6.5 and 7.5.

TABLE 3
Composition of 10% Ascorbic Acid Derivative Compositions with Different pH

| Effect of pH on viscosity and | | | | | | | |
|---|---|---|---|---|---|---|---|
| stability of compositions | | | FL# 5 | FL# 6 | FL# 7 | FL# 8 | FL# 9 |
| Function | Trade Name | US INCI | RM % | RM % | RM % | RM % | RM % |
| Solvent | DEIONIZED | WATER | 59.54 | 59.89 | 60.29 | 60.84 | 61.19 |
| | WATER | | | | | | |
| Chelating | BIOPURE ™ | WATER, TETRASODIUM | 0.20 | 0.20 | 0.20 | 0.20 | 0.20 |
| | GLDA | GLUTAMATE DIACETATE, | | | | | |
| | | SODIUM HYDROXIDE | | | | | |
| Antioxidant | SODIUM | SODIUM | 0.01 | 0.01 | 0.01 | 0.01 | 0.01 |
| | METABISULFITE | METABISULFITE | | | | | |
| Humectants | GLYCERINE | GLYCERIN | 3.00 | 3.00 | 3.00 | 3.00 | 3.00 |
| | 99.7%/MB | | | | | | |
| | ZEMEA | PROPANEDIOL | 10.00 | 10.00 | 10.00 | 10.00 | 10.00 |
| Preservative | PHENOXETOL | PHENOXYETHANOL | 0.90 | 0.90 | 0.90 | 0.90 | 0.90 |
| | NF 272556 | | | | | | |
| Thickener | SILIGEL ™ | XANTHAN GUM, | 0.30 | 0.30 | 0.30 | 0.30 | 0.30 |
| | | SCLEROTIUM | | | | | |
| | | GUM, LECITHIN, | | | | | |
| | | PULLULAN, SILICA | | | | | |
| O/W | NET-WRS | SORBETH-30 | 2.50 | 2.50 | 2.50 | 2.50 | 2.50 |
| Emulsifier | | TETRAISOSTEARATE, | | | | | |
| | | WATER, DIPROPYLENE | | | | | |
| | | GLYCOL, SORBITAN | | | | | |
| | | SESQUIISOSTEARATE, | | | | | |
| | | ACRYLATES/BEHENETH-25 | | | | | |
| | | METHACRYLATE COPOLYMER, | | | | | |
| | | PPG-8-CETETH-20, | | | | | |
| | | PHENOXYETHANOL | | | | | |
| Oil | NEOLIGHT 100P | ISODECYL | 3.00 | 3.00 | 3.00 | 3.00 | 3.00 |
| | | NEOPENTANOATE | | | | | |
| | LEXFEEL 7 | NEOPENTYL GLYCOL | 2.00 | 2.00 | 2.00 | 2.00 | 2.00 |
| | | DIHEPTANOATE | | | | | |
| | VEGELIGHT | C9-12 ALKANE, COCO- | 2.00 | 2.00 | 2.00 | 2.00 | 2.00 |
| | C912-LC | CAPRYLATE/CAPRATE | | | | | |
| | NEOSSANCE ™ | SQUALANE | 3.00 | 3.00 | 3.00 | 3.00 | 3.00 |
| | SQUALANE | | | | | | |
| | VITAMIN E- | TOCOPHERYL | 0.50 | 0.50 | 0.50 | 0.50 | 0.50 |
| | ACETATE CARE | ACETATE | | | | | |
| | 30499500 | | | | | | |
| Buffer/pH | SODIUM | SODIUM | 0.080 | 0.080 | 0.080 | 0.080 | 0.080 |
| adjust | CITRATE | CITRATE | | | | | |
| | CITRIC ACID | CITRIC ACID | 1.720 | 1.370 | 0.970 | 0.420 | 0.070 |
| Ascorbic | Ascorbyl | Ascorbyl | 1.000 | 1.000 | 1.000 | 1.000 | 1.000 |
| acid | Glucoside | Glucoside | | | | | |
| derivatives | Sodium Ascorbyl | Sodium Ascorbyl | 9.000 | 9.000 | 9.000 | 9.000 | 9.000 |
| | Phosphate | Phosphate | | | | | |
| Polymer/ | Aristoflex Silk | Sodium Polyacryloyldimethyl | 1.250 | 1.250 | 1.250 | 1.250 | 1.250 |
| thickener | | Taurate | | | | | |

| | | | | | |
|---|---|---|---|---|---|
| Total | 100.0000 | 100.0000 | 100.0000 | 100.0000 | 100.0000 |

TABLE 4
Viscosity and Stability Comparison

| | | | Stability |
|---|---|---|---|
| | | Viscosity | 2 |
| | pH | Cps | weeks/@RT |
| FL # 5 | 5.59 | 4450 | Separation |
| FL # 6 | 6.00 | 7200 | Separation |
| FL # 7 | 6.60 | 7750 | Stable |
| FL # 8 | 7.00 | 7850 | Stable |
| FL # 9 | 7.40 | 8100 | Stable |

The compositions #5, 6, 7, 8 and 9 were subjected to aging by placing them in an oven at 0° C., 25° C., 37° C. and 40° C.) for four weeks. Pictures were taken at different stages from initial, 2 weeks and 4 weeks. The degradation was evaluated in color change by visual inspection.

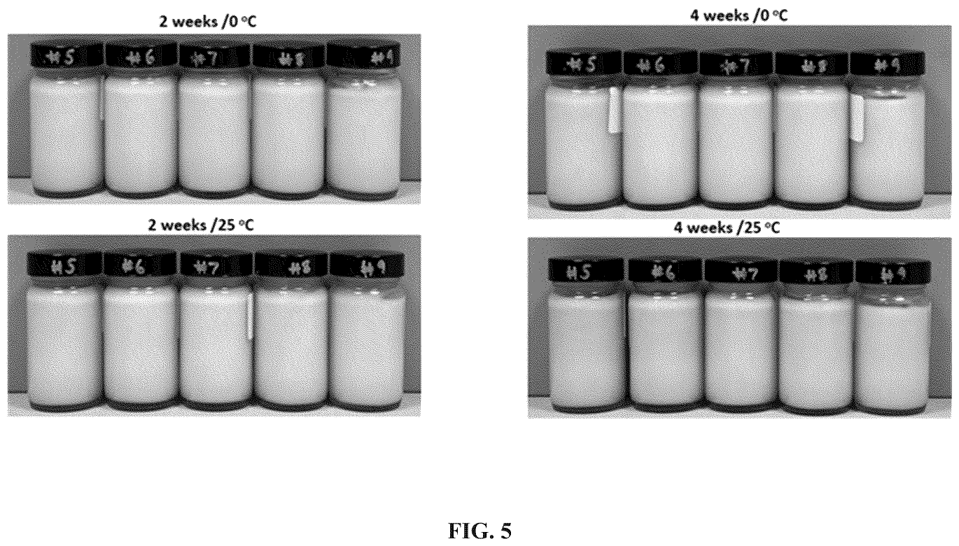

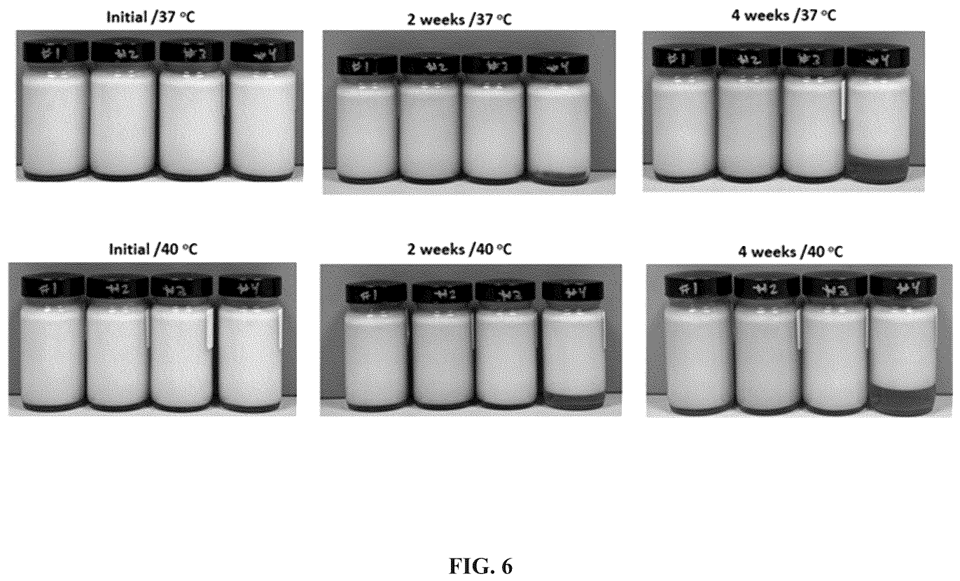

FIGS. 5 and 6 show the color and appearance changes in different temperature and over time. FIG. 5 is photos illustrating color change of compositions at 0° C. and 25° C. over time. As shown in FIG. 5, the only discoloration (creamy white to brown/orange) for any of the samples occurred at 4 weeks at 25° C. with very slight discoloration occurring for each composition #5, #6, #7, #7, and #8. FIG. 6 is photos illustrating color change of compositions at 37° C. and 40° C. over time.

All the compositions showed color changes by increasing temperature, for example at 40° C. Compositions having higher pH, such as FL #8 and FL #9, showed good stability with less color change than the other compositions of this example. Compositions having pH<6.0 such as FL #5 and FL #6 showed separation with more color change than the other composition of this example.

Optimum pH range for stabilizing 10% ascorbic acid derivative composition was shown to be 6.5-7.5.

In addition, an assay of SAP and AA2G were analyzed by HPLC. The result is listed in Table 5. Table 5 showed that chemical stability of FL #1 is relatively stable, even at 45° C. for 4 weeks.

TABLE 5
Ascorbic Acid Derivative Loss % of Inventive (FL#1) Over Time

| | % Loss | % Loss | % Loss | % Loss |
|---|---|---|---|---|
| | @25° C. | @37° C. | @40° C. | @45° C. |
| (FL#1) | (4 weeks) | (4 weeks) | (4 weeks) | (4 weeks) |
| SAP (9%) | 0 | 0 | −1.22 | −2.56 |
| AA2G (1%) | −1.00 | −1.00 | −1.00 | 0 |

### Compositions Example 3

The thickener selection has a significant impact on the ascorbic acid derivative stability of the compositions. Aristoflex Silk was identified as a key thickener to stabilize the multi-derivative compositions. The concentration of Aristoflex Silk was varied to determine the optimal range for stability and viscosity. The compositions are listed in Table 6 including control FL #4.

TABLE 6
Composition of 10% Ascorbic Acid Derivative Compositions with Different Concentration of Aristoflex Silk

| Effect of Aristoflex Silk % on viscosity | | | | | | | | |
|---|---|---|---|---|---|---|---|---|
| and stability of compositions | | | FL# 4 | FL# 10 | FL# 11 | FL# 1 | FL# 13 | FL# 14 |
| Function | Trade Name | US INCI | RM % | RM % | RM % | RM % | RM % | RM % |
| Solvent | DEIONIZED WATER | WATER | 61.78 | 61.05 | 60.89 | 60.64 | 60.39 | 60.20 |
| Chelating | BIOPURE ™ GLDA | WATER, TETRASODIUM | 0.20 | 0.20 | 0.20 | 0.20 | 0.20 | 0.20 |
| | | GLUTAMATE DIACETATE, | | | | | | |
| | | SODIUM HYDROXIDE | | | | | | |
| Antioxidant | SODIUM | SODIUM | 0.01 | 0.01 | 0.01 | 0.01 | 0.01 | 0.01 |
| | METABISULFITE | METABISULFITE | | | | | | |
| Humectants | GLYCERINE | GLYCERIN | 3.00 | 3.00 | 3.00 | 3.00 | 3.00 | 3.00 |
| | 99.7%/MB | | | | | | | |
| | ZEMEA | PROPANEDIOL | 10.00 | 10.00 | 10.00 | 10.00 | 10.00 | 10.00 |
| Preservative | PHENOXETOL NF | PHENOXYETHANOL | 0.90 | 0.90 | 0.90 | 0.90 | 0.90 | 0.90 |
| | 272556 | | | | | | | |
| Thickener | SILIGEL ™ | XANTHAN GUM, | 0.30 | 0.30 | 0.30 | 0.30 | 0.30 | 0.30 |
| | | SCLEROTIUM | | | | | | |
| | | GUM, LECITHIN, | | | | | | |
| | | PULLULAN, SILICA | | | | | | |
| O/W | NET-WRS | SORBETH-30 | 2.50 | 2.50 | 2.50 | 2.50 | 2.50 | 2.50 |
| Emulsifier | | TETRAISOSTEARATE, | | | | | | |
| | | WATER, DIPROPYLENE | | | | | | |
| | | GLYCOL, SORBITAN | | | | | | |
| | | SESQUIISOSTEARATE, | | | | | | |
| | | ACRYLATES/BEHENETH-25 | | | | | | |
| | | METHACRYLATE COPOLYMER, | | | | | | |
| | | PPG-8-CETETH-20, | | | | | | |
| | | PHENOXYETHANOL | | | | | | |
| Oil | NEOLIGHT 100P | ISODECYL NEOPENTANOATE | 3.00 | 3.00 | 3.00 | 3.00 | 3.00 | 3.00 |
| | LEXFEEL 7 | NEOPENTYL GLYCOL | 2.00 | 2.00 | 2.00 | 2.00 | 2.00 | 2.00 |
| | | DIHEPTANOATE | | | | | | |
| | VEGELIGHT C912- | C9-12 ALKANE, COCO- | 2.00 | 2.00 | 2.00 | 2.00 | 2.00 | 2.00 |
| | LC | CAPRYLATE/CAPRATE | | | | | | |
| | NEOSSANCE ™ | SQUALANE | 3.00 | 3.00 | 3.00 | 3.00 | 3.00 | 3.00 |
| | SQUALANE | | | | | | | |
| | VITAMIN E- | TOCOPHERYL | 0.50 | 0.50 | 0.50 | 0.50 | 0.50 | 0.50 |
| | ACETATE CARE | ACETATE | | | | | | |
| | 30499500 | | | | | | | |
| Buffer/pH | SODIUM | SODIUM | 0.080 | 0.080 | 0.080 | 0.080 | 0.080 | 0.080 |
| adjust | CITRATE | CITRATE | | | | | | |
| | CITRIC ACID | CITRIC ACID | 0.730 | 0.710 | 0.625 | 0.625 | 0.625 | 0.560 |
| Ascorbic | Ascorbyl | Ascorbyl | 1.000 | 1.000 | 1.000 | 1.000 | 1.000 | 1.000 |
| acid | Glucoside | Glucoside | | | | | | |
| derivatives | Sodium Ascorbyl | Sodium Ascorbyl | 9.000 | 9.000 | 9.000 | 9.000 | 9.000 | 9.000 |
| | Phosphate | Phosphate | | | | | | |
| Polymer/ | Aristoflex Silk | Sodium Polyacryloyldimethyl | 0.000 | 0.750 | 1.000 | 1.250 | 1.500 | 1.750 |
| thickener | | Taurate | | | | | | |

| | | | | | | |
|---|---|---|---|---|---|---|
| Total | 100.000 | 100.000 | 100.000 | 100.000 | 100.000 | 100.000 |

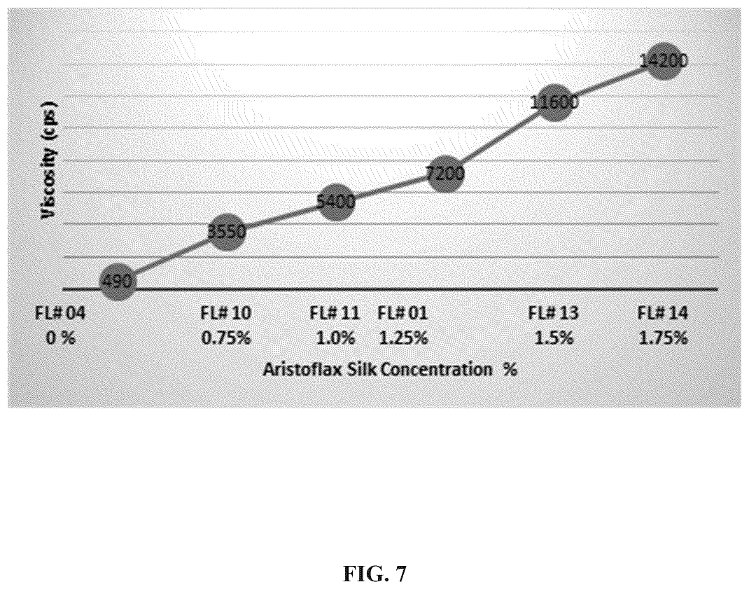

FIG. 7 is a graph illustrating viscosity increase with concentration increase of Aristoflex Silk. Table 7 shows that the stability of the FL #1, #11, #13 and #14 are good. FL #14 has the highest viscosity among all of the compositions and has the best stability even in the harsh conditions @3 cys F/T. Optimal range of Aristoflex Silk was found to be 1.25% to 1.75% for the good stability. FL #1 showed desired viscosity.

TABLE 7
Viscosity and Stability Comparison for FL
Containing Different Aristoflex Silk %

| | Aristoflex | Viscosity | 4 weeks | 4 weeks | 3cys |
|---|---|---|---|---|---|
| Formula# | % | (Cps) | @25° C. | @40° C. | @F/T |
| FL# 04 | 0 | 490 | unstable | unstable | unstable |
| FL# 10 | 0.75 | 3550 | unstable | unstable | unstable |
| FL# 11 | 1.0 | 5400 | Stable | Stable | unstable |
| FL# 01 | 1.25 | 7200 | Stable | Stable | Stable |
| FL# 13 | 1.5 | 11600 | Stable | Stable | Stable |
| FL# 14 | 1.75 | 14200 | Stable | Stable | Stable |

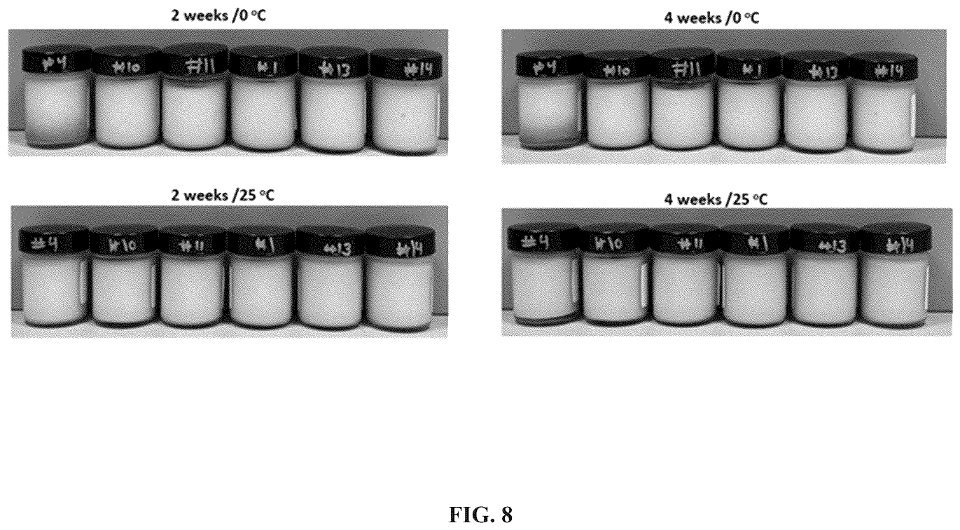

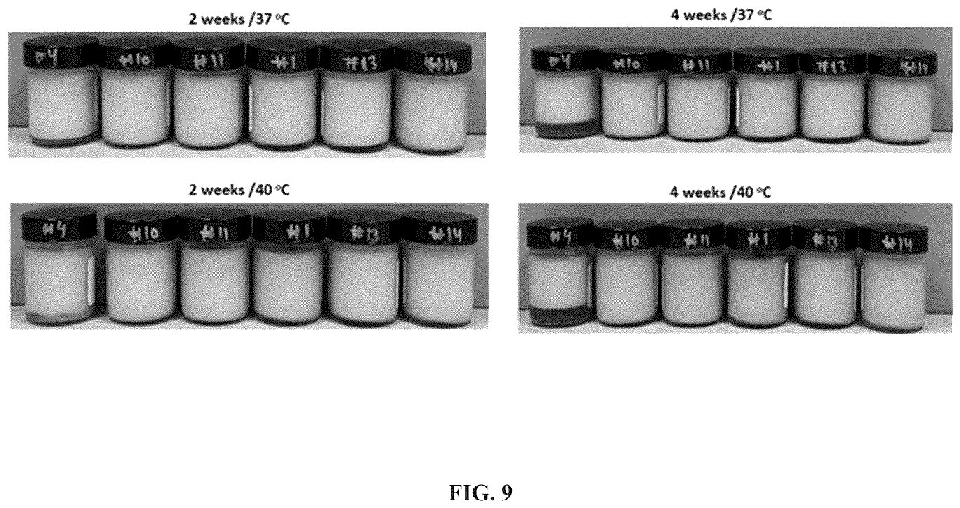

The compositions FL #1, #4, #10, #11, #13 and #14 were subjected to aging by placing them in an oven at 0° C., 25° C., 37° C. and 40° C.) for four weeks. Pictures were taken at different stages of 2 weeks and 4 weeks. The degradation was evaluated in color change by visual inspection. FIGS. 8 and 9 illustrate the color and appearance changes at different temperatures over time. All the compositions showed color changes by increasing temperature, for example, at 40° C. FLs having higher pH, such as FL #8 and FL #9, show good stability with less color change. FLs having pH<6.0, such as FL #5 and FL #6, showed separation with more color change. Optimal pH range for stabilizing 10% ascorbic acid derivative composition (9% SAP and 1% AA2G) was found to be 6.5-7.5. Aristoflex Silk concentration of 1.25% to 1.75% showed the best stability tested, and FL #1 provided the best viscosity tested.

### Compositions Example 4

Aristoflex Silk was identified as a key thickener to stabilize the compositions. The emulsifier NET-WRS was also important to stabilize the compositions and also provide the unique watery and nice texture. 1.25% Aristoflex Silk was used in each composition (FL #1, #15, #16, #17, #18, #19). The compositions are listed in Table 8 including control FL #15.

TABLE 8
Composition of 10% Ascorbic Acid Derivatives Compositions with Different Concentration of NET-WRS

| Effect of ratio of NET-WRS to Aristoflex Silk | | | | | | | | | |
|---|---|---|---|---|---|---|---|---|---|
| on viscosity and stability of compositions | | | FL# 15 | FL# 16 | FL# 17 | FL# 1 | FL# 18 | FL# 19 | FL# 20 |
| Function | Trade Name | US INCI | RM % | RM % | RM % | RM % | RM % | RM % | RM % |
| Solvent | DEIONIZED | WATER | 63.13 | 62.16 | 61.11 | 60.51 | 60.01 | 59.01 | 58.03 |
| | WATER | | | | | | | | |
| Chelating | BIOPURE ™ | WATER, TETRASODIUM | 0.20 | 0.20 | 0.20 | 0.20 | 0.20 | 0.20 | 0.20 |
| | GLDA | GLUTAMATE DIACETATE, | | | | | | | |
| | | SODIUM HYDROXIDE | | | | | | | |
| Antioxidant | SODIUM | SODIUM | 0.01 | 0.01 | 0.01 | 0.01 | 0.01 | 0.01 | 0.01 |
| | METABISULFITE | METABISULFITE | | | | | | | |
| Humectants | GLYCERINE | GLYCERIN | 3.00 | 3.00 | 3.00 | 3.00 | 3.00 | 3.00 | 3.00 |
| | 99.7%/MB | | | | | | | | |
| | ZEMEA | PROPANEDIOL | 10.00 | 10.00 | 10.00 | 10.00 | 10.00 | 10.00 | 10.00 |
| Preservative | PHENOXETOL | PHENOXYETHANOL | 0.90 | 0.90 | 0.90 | 0.90 | 0.90 | 0.90 | 0.90 |
| | NF 272556 | | | | | | | | |
| Thickener | SILIGEL ™ | XANTHAN GUM, | 0.30 | 0.30 | 0.30 | 0.30 | 0.30 | 0.30 | 0.30 |
| | | SCLEROTIUM | | | | | | | |
| | | GUM, LECITHIN, | | | | | | | |
| | | PULLULAN, SILICA | | | | | | | |
| O/W | NET-WRS | SORBETH-30 | 0.00 | 1.00 | 2.00 | 2.50 | 3.00 | 4.00 | 5.00 |
| Emulsifier | | TETRAISOSTEARATE, | | | | | | | |
| | | WATER, DIPROPYLENE | | | | | | | |
| | | GLYCOL, SORBITAN | | | | | | | |
| | | SESQUIISOSTEARATE, | | | | | | | |
| | | ACRYLATES/BEHENETH-25 | | | | | | | |
| | | METHACRYLATE COPOLYMER, | | | | | | | |
| | | PPG-8-CETETH-20, | | | | | | | |
| | | PHENOXYETHANOL | | | | | | | |
| Oil | NEOLIGHT 100P | ISODECYL | 3.00 | 3.00 | 3.00 | 3.00 | 3.00 | 3.00 | 3.00 |
| | | NEOPENTANOATE | | | | | | | |
| | LEXFEEL 7 | NEOPENTYL GLYCOL | 2.00 | 2.00 | 2.00 | 2.00 | 2.00 | 2.00 | 2.00 |
| | | DIHEPTANOATE | | | | | | | |
| | VEGELIGHT | C9-12 ALKANE, | 2.00 | 2.00 | 2.00 | 2.00 | 2.00 | 2.00 | 2.00 |
| | C912-LC | COCO-CAPRYLATE/ | | | | | | | |
| | | CAPRATE | | | | | | | |
| | NEOSSANCE ™ | SQUALANE | 3.00 | 3.00 | 3.00 | 3.00 | 3.00 | 3.00 | 3.00 |
| | SQUALANE | | | | | | | | |
| | VITAMIN E- | TOCOPHERYL | 0.50 | 0.50 | 0.50 | 0.50 | 0.50 | 0.50 | 0.50 |
| | ACETATE CARE | ACETATE | | | | | | | |
| | 30499500 | | | | | | | | |
| Buffer/pH | SODIUM | SODIUM | 0.080 | 0.080 | 0.080 | 0.080 | 0.080 | 0.080 | 0.080 |
| adjust | CITRATE | CITRATE | | | | | | | |
| | CITRIC ACID | CITRIC ACID | 0.630 | 0.600 | 0.650 | 0.750 | 0.750 | 0.750 | 0.730 |
| Ascorbic | Ascorbyl | Ascorbyl | 1.000 | 1.000 | 1.000 | 1.000 | 1.000 | 1.000 | 1.000 |
| acid | Glucoside | Glucoside | | | | | | | |
| derivatives | Sodium | Sodium | 9.000 | 9.000 | 9.000 | 9.000 | 9.000 | 9.000 | 9.000 |
| | Ascorbyl | Ascorbyl | | | | | | | |
| | Phosphate | Phosphate | | | | | | | |
| Polymer/ | Aristoflex Silk | Sodium Polyacryloyldimethyl | 1.250 | 1.250 | 1.250 | 1.250 | 1.250 | 1.250 | 1.250 |
| thickener | | Taurate | | | | | | | |

| | | | | | | | |
|---|---|---|---|---|---|---|---|
| Total | 100.000 | 100.000 | 100.000 | 100.00 | 100.000 | 100.00 | 100.000 |

The emulsifier NET-WRS concentration was varied in each composition to determine the optimal range for stability and viscosity. The pH of the compositions remained the same at 6.9. The viscosity and stability are shown in Table 9.

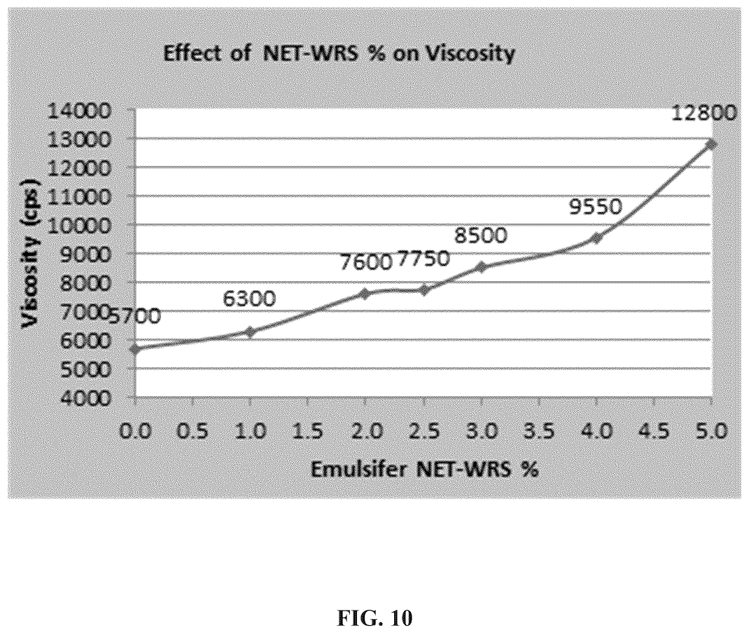

The thickening efficiency is compared in FIG. 10. As can be seen, the emulsifier NET-WRS has an important effect on the stability of the ascorbic acid derivative compositions. Table 9 shows that NET-WRS>2.0 showed good thickening effect, as well as good stability at harsh conditions (3 cycles of Freeze/Thaw).

TABLE 9
Effect of NET-WRS % on Viscosity and Stability

| | | Viscosity | Stability | |
|---|---|---|---|---|
| | NET-WRS | (cps) | 4 | Stability |
| Composition # | % | overnight | weeks/@RT | 3 cys/@F/T |
| FL# 15 | 0.0 | 5700 | OK | Separate |
| FL# 16 | 1.0 | 6300 | Stable | Separate |
| FL# 17 | 2.0 | 7600 | Stable | Separate |
| FL#01 | 2.5 | 7750 | Stable | Good |
| FL# 18 | 3.0 | 8500 | Stable | Good |
| FL# 19 | 4.0 | 9550 | Stable | Good |
| FL# 20 | 5.0 | 12800 | Stable | Good |

### Compositions Example 5

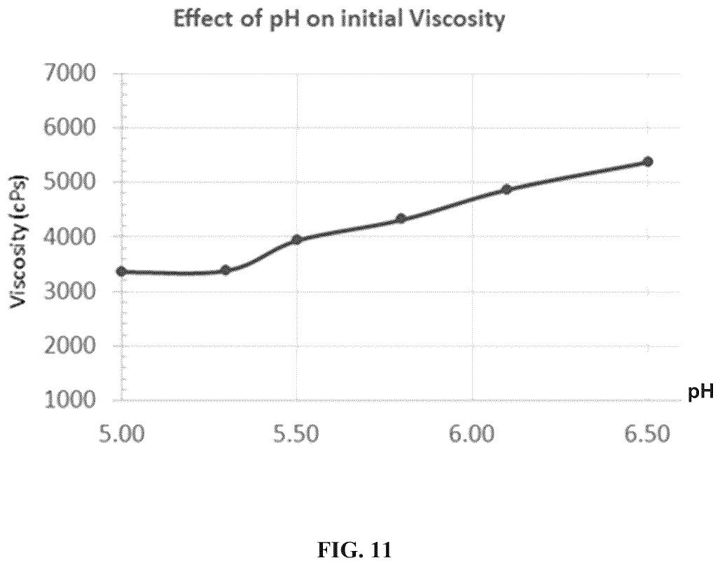

The pH value has significant impact on the ascorbic acid derivative stability of the compositions. Six compositions #21, #22, #23, #24, #25, and #26 were prepared with the same ingredients and varied pH values from 5.0 to 6.5 adjusted by citric acid. The compositions are listed in Table 10. The viscosity and stability comparison are shown in Table 10 and FIG. 11.

TABLE 10
Composition of 10% Ascorbic Acid Derivative Compositions with 3 Ascorbic Acid Derivatives with Varying pH

| Effects of pH on viscosity and | | | FL# 21 | FL# 22 | FL# 23 | FL# 24 | FL# 25 | FL# 26 |
|---|---|---|---|---|---|---|---|---|
| stability of compositions | | | pH = | pH = | pH = | pH = | pH = | pH = |
| Function | Trade Name | INCI Name | 5.03 | 5.33 | 5.56 | 5.77 | 6.09 | 6.54 |
| Solvent | DEIONIZED | WATER | 61.100 | 61.305 | 61.490 | 61.610 | 61.800 | 62.080 |
| | WATER | | | | | | | |
| Chelating | BIOPURE ™ | WATER & TETRASODIUM | 0.200 | 0.200 | 0.200 | 0.200 | 0.200 | 0.200 |
| | GLDA | GLUTAMATE DIACETATE & | | | | | | |
| | | SODIUM HYDROXIDE | | | | | | |
| Humectant | ZEMEA | PROPANEDIOL | 9.000 | 9.000 | 9.000 | 9.000 | 9.000 | 9.000 |
| | PROPANEDIOL | | | | | | | |
| Humectant | GLYCERINE | GLYCERIN | 3.000 | 3.000 | 3.000 | 3.000 | 3.000 | 3.000 |
| | 99.7%/MB | | | | | | | |
| Preservative | PHENOXETOL | PHENOXYETHANOL | 0.900 | 0.900 | 0.900 | 0.900 | 0.900 | 0.900 |
| | NF 272556 | | | | | | | |
| Antioxidant | SODIUM | SODIUM METABISULFITE | 0.010 | 0.010 | 0.010 | 0.010 | 0.010 | 0.010 |
| | PYROSULFITE | | | | | | | |
| Thickener | ARISTOFLEX ® | SODIUM | 1.250 | 1.250 | 1.250 | 1.250 | 1.250 | 1.250 |
| | SILK | POLYACRYLOYLDIMETHYL | | | | | | |
| | | TAURATE | | | | | | |
| O/W | NET-WRS | SORBETH-30 | 2.500 | 2.500 | 2.500 | 2.500 | 2.500 | 2.500 |
| Emulsifier | | TETRAISOSTEARATE, | | | | | | |
| | | WATER, DIPROPYLENE | | | | | | |
| | | GLYCOL, SORBITAN | | | | | | |
| | | SESQUIISOSTEARATE & | | | | | | |
| | | ACRYLATES/BEHENETH-25 | | | | | | |
| | | METHACRYLATE COPOLYMER, | | | | | | |
| | | PPG-8-CETETH-20, | | | | | | |
| | | PHENOXYETHANOL | | | | | | |
| Solvent | BOTANESSENTIAL | ETHYLHEXYL | 10.000 | 10.000 | 10.000 | 10.000 | 10.000 | 10.000 |
| | OLIVE S-EHO | OLIVATE & SQUALANE | | | | | | |
| Antioxidant | VITAMIN E- | TOCOPHERYL | 0.500 | 0.500 | 0.500 | 0.500 | 0.500 | 0.500 |
| | ACETATE CARE | ACETATE | | | | | | |
| | 30499500 | | | | | | | |
| Vitamin C | AA2G ™ | ASCORBYL GLUCOSIDE | 1.000 | 1.000 | 1.000 | 1.000 | 1.000 | 1.000 |
| derivatives | | | | | | | | |
| | SODIUM | SODIUM | 6.000 | 6.000 | 6.000 | 6.000 | 6.000 | 6.000 |
| | ASCORBYL | ASCORBYL | | | | | | |
| | PHOSPHATE | PHOSPHATE | | | | | | |
| | RONACARE ® | MAGNESIUM | 3.000 | 3.000 | 3.000 | 3.000 | 3.000 | 3.000 |
| | MAP | ASCORBYL | | | | | | |
| | | PHOSPHATE | | | | | | |
| Buffer/pH | SODIUM | SODIUM | 0.080 | 0.080 | 0.080 | 0.080 | 0.080 | 0.080 |
| Adjuster | CITRATE | CITRATE | | | | | | |
| | ANHYDROUS | | | | | | | |
| | GRANU | | | | | | | |
| | CITRIC ACID | CITRIC ACID | 1.460 | 1.255 | 1.070 | 0.950 | 0.760 | 0.480 |
| | ANHYDROUS | | | | | | | |
| | | | 100.0000 | 100.0000 | 100.0000 | 100.0000 | 100.0000 | 100.0000 |

FIG. 11 shows that viscosity increased with increased pH value. There was no recrystallization observed in FL #23 and #24 after 8 weeks. Recrystallization occurred in FL #25 and FL #26 (pH>6). Table 11 shows that optimal pH range for stabilizing the FL is between 5.5 and 5.8.

TABLE 11
Viscosity and Stability Comparison

| | | Viscosity | Stability 8 weeks |
|---|---|---|---|
| | pH | cPs | @ RT |
| FL# 21 | 5.00 | 3350 | Separation |
| FL# 22 | 5.30 | 3380 | Separation |
| FL# 23 | 5.50 | 3930 | Stable |
| FL# 24 | 5.80 | 4310 | Stable |
| FL# 25 | 6.10 | 4860 | Recrystallization |
| FL# 26 | 6.50 | 5360 | Recrystallization |

The compositions (FL) #21, #22, #23, #24, #25 and #26 were subjected to aging by placing them in an oven at −5° C., 25° C., 37° C., 40° C. and 45° C.) for four weeks. Pictures were taken at different stages at 2 weeks and 4 weeks. The degradation was evaluated in color change by visual inspection.

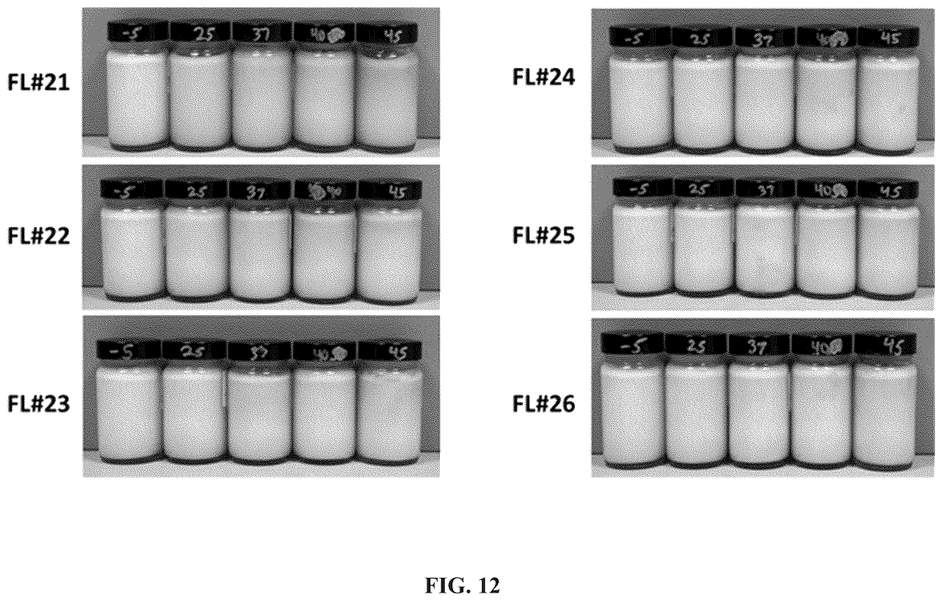

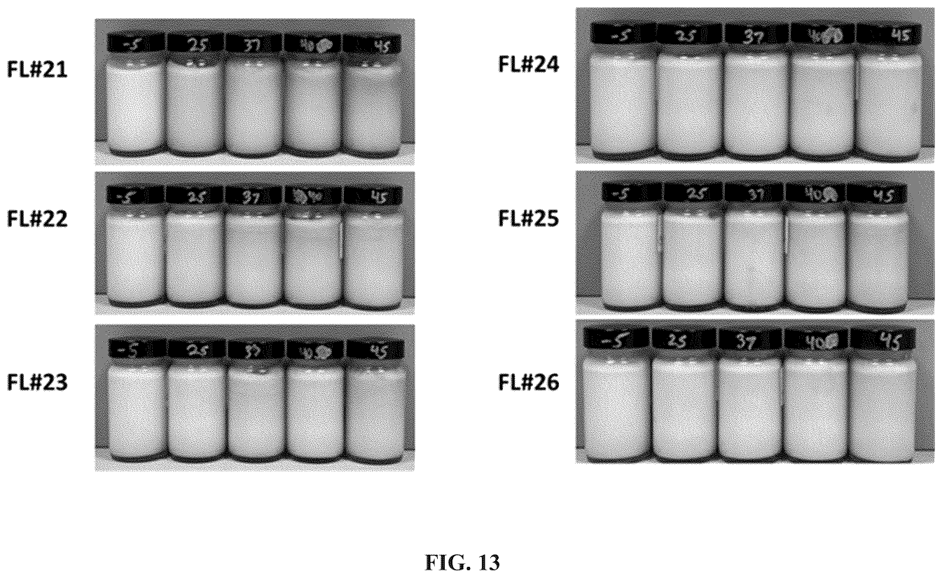

FIGS. 12 and 13 show the color and appearance changes at different temperature and over time. All the compositions showed color changes by increasing temperature, for example at 45° C. In addition, all of the compositions showed increased discoloration (creamy white to increased amount of brown/orange) over time (2 weeks of FIG. 12 versus 4 weeks of FIG. 13). The compositions having pH<5.5, such as FL #21 and #22, showed phase separation. The compositions having pH>6.8, such as FL #25 and #26, showed recrystallization occurring after 8 weeks (not shown). The compositions having pH 5.5-5.8, such as FL #23 and #24, showed good stability with less color change, even at 45° C., as compared to the other compositions of this example. Optimal pH range for stabilizing 10% multi-derivative composition (6% SAP, 1% AA2G and 3% MAP) was found to be 5.5-5.8.

Overall, compositions of ascorbic acid derivatives of the present disclosure can be oil-in-water emulsions including two or more ascorbic acid derivatives, an emulsifier, and a functional polymer. Compositions of the present disclosure can be emulsions that are storage stable for extended periods of time with reduced or eliminated degradation and/or crystallization of the ascorbic acid-based compounds while maintaining or improving the efficacy of the ascorbic acid-based compounds. Compositions of the present disclosure can be salt-tolerant and have efficacious amounts of ascorbic acid derivatives (e.g., 10 wt % or greater of ascorbic acid derivatives) without degradation of the ascorbic acid derivative(s) but with maintained stabilization of an oil-in-water emulsion (e.g., substantially no or no biphasic separation of the emulsion) and without crystallization (localized hot spots) of ascorbic acid derivative(s) during use.

In addition, compositions of the present disclosure can optionally be substantially free of or entirely free of silicone, polyethylene glycol, alcohol, fragrance, or combinations thereof. Thus, when applied to the skin, component(s) of compositions of the present disclosure can evaporate from the skin without leaving a waxy or oil residue left behind.

Unless otherwise specified “a” or “an” means one or more.

As used herein, the term “about” placed before a specific numeric value may mean plus or minus 20 percent of the numeric value; plus or minus 18 percent of the numeric value, plus or minus 15 percent of the numeric value; plus or minus 12 percent of the numeric value; plus or minus 8 percent of the numeric value; plus or minus 5 percent of the numeric value; plus or minus 3 percent of the numeric value; plus or minus 2 percent of the numeric value; plus or minus 1 percent of the numeric value or plus or minus 0.5 percent of the numeric value. All content information for ingredients of compositions expressed as percent (percent) refers to percent (percent) by mass, relative to the total mass of the composition, unless specified otherwise.

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the present disclosure, additionally, they do not exclude impurities and variances normally associated with the elements and materials used.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All documents described herein are incorporated by reference herein, including any priority documents and or testing procedures to the extent they are not inconsistent with this text. As is apparent from the foregoing general description and the specific embodiments, while forms of the present disclosure have been illustrated and described, various modifications can be made without departing from the spirit and scope of the present disclosure. Accordingly, it is not intended that the present disclosure be limited thereby. Likewise, the term “comprising” is considered synonymous with the term “including” for purposes of United States law. Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

While the present disclosure has been described with respect to a number of embodiments and examples, those skilled in the art, having benefit of this disclosure, will appreciate that other embodiments can be devised which do not depart from the scope and spirit of the present disclosure.

1. A composition comprising:
water;
an oil;
two or more ascorbic acid derivatives in a total amount of about 9 wt % to about 15 wt %, wherein the ascorbic acid derivatives are selected from the group consisting of sodium ascorbyl phosphate (SAP), magnesium ascorbyl phosphate (MAP), and ascorbyl glucoside (AA2G);
an emulsifier; and
a functional polymer.

2. The composition of claim 1, wherein the composition comprises the water in an amount of about 55 wt % to about 65 wt % and the oil in an amount of about 7 wt % to about 13 wt %.

3. The composition of claim 1, wherein the total amount of the two or more ascorbic acid derivatives is about 9 wt % to about 11 wt %.

4. The composition of claim 1, wherein the two or more ascorbic acid derivatives are SAP and AA2G, wherein SAP is present in an amount of about 8 wt % to about 10 wt % and AA2G is present in an amount of about 0.5 wt % to about 3 wt %.

5. The composition of claim 4, wherein the composition has a pH of about 6.5 to about 7.5.

6. The composition of claim 5, wherein the composition has about 2 wt % to about 4 wt % isodecyl neopentanoate, about 1 wt % to about 3 wt % neopentyl glycol diheptanoate, about 0.01 wt % to about 1.1 wt % coco-caprylate/caprate, about 2 wt % to about 4 wt % squalene, and about 0.3 wt % to about 1 wt % tocopheryl acetate.

7. The composition of claim 1, wherein the two or more ascorbic acid derivatives are each SAP, AA2G, and MAP, wherein SAP is present in an amount of about 5 wt % to about 7 wt %, AA2G is present in an amount of about 0.5 wt % to about 3 wt %, and MAP is present in an amount of about 2 wt % to about 3.5 wt %.

8. The composition of claim 7, wherein the composition has a pH of about 5.5 to about 5.8.

9. The composition of claim 8, wherein the composition comprises about 7 wt % to about 13 wt % squalene plus ethylhexyl olivate.

10. The composition of claim 1, wherein the functional polymer is present in an amount of about 1.25 wt % to about 1.75 wt %.

11. The composition of claim 9, wherein the emulsifier is present in an amount of about 2.5 wt % to about 5 wt %.

12. The composition of claim 1, wherein the composition comprises about 1 wt % or less of silicone, polyethylene glycol, alcohol or their combinations thereof.

13. The composition of claim 1, wherein the emulsifier comprises a copolymer of (1) acrylate or methacrylate monomeric units and (2) a polyethylene glycol ether.

14. The composition of claim 13, wherein the copolymer is a copolymer of an ester of methacrylic acid and the polyethylene glycol ether.

15. The composition of claim 14, wherein the emulsifier comprises the copolymer, a stearate, a glycol, a polypropylene glycol copolymer, and a phenoxyalcohol.

16. The composition of claim 15, wherein the emulsifier comprises the copolymer, a tetraisostearate, dipropylene glycol, sorbitan sesquiisostearate, the polypropylene glycol copolymer, and a phenoxyethanol.

17. The composition of claim 16, wherein the polypropylene glycol copolymer comprises a polyethylene glycol ether of sorbitol with an average of about 30 moles of ethylene oxide.

18. The composition of claim 13, wherein the functional polymer comprises a polyacryloyldimethyl taurate.

19. The composition of claim 18, wherein the polyacryloyldimethyl taurate comprises a sodium polyacryloyldimethyl taurate.

20. The composition of claim 1, wherein the composition is oil-in-water emulsion.

21. A method comprising treating skin by applying the composition according to claim 1 to the skin.

22. The composition of claim 1, wherein the composition further comprises:
a chelating agent in an amount of about 0.1 wt % to about 0.5 wt %;
a humectant in an amount of about 2 wt % to about 4 wt %, about 9 wt % to about 11 wt %, or about 12 wt % to about 14 wt %;
a pH adjuster in an amount of about 0.5 wt % to about 1 wt % or about 1.5 wt % to about 2 wt %;
an antioxidant in an amount of about 0.1 wt % to about 1.0 wt %; and
a preservative in an amount of about 0.5 wt % to about 1.5 wt %.

23. A method comprising treating skin by applying the composition according to claim 22.
